(19) United States
(12) Patent Application Publication
(10) Pub. No.: US 2026/0356981 A1
(43) Pub. Date: Nov. 12, 2026

(54) PROCESS OF PURIFICATION OF PROTEIN

(71) Applicant: KASHIV BIOSCIENCES, LLC, Piscataway, NJ (US)

(72) Inventors: Tarun Kumar Gupta, Ahmedabad (IN); Mayankkumar Thakkar, Ahmedabad (IN)

(21) Appl. No.: 19/746,318

(22) Filed: Jul. 17, 2026

Related U.S. Application Data
(63) Continuation of application No. 19/731,424, filed on Jul. 6, 2026.
Application No. 19/731,424 is a continuation-in-part of application No. 19/432,924, filed on Dec. 24, 2025.
Application No. 19/432,924 is a continuation-in-part of application No. 17/922,729, filed on May 1, 2021.

(30) Foreign Application Priority Data
May 1, 2020 (IN) 202021018714
May 1, 2020 (IN) 202021018731
May 1, 2020 (IN) 202021018735

(51) Int. Cl. C07K 16/42; A61K 47/02; A61K 47/12; A61K 47/22

(52) U.S. Cl. CPC C07K 16/4291; A61K 47/02; A61K 47/12; A61K 47/22; C07K 2317/24

(57) ABSTRACT

The present invention provides a drug substance composition comprising a biosimilar of omalizumab, having a defined levels of acidic and basic variants, High molecular weight impurities (HMW), Light molecular weight impurities (LMW) and suitable amount of light chain and heavy chain showing stability under stress conditions for three days, seven days, fifteen days and thirteen days.

### CROSS-REFERENCE TO RELATED APPLICATIONS

This application is a continuation of continuation-in-part U.S. patent application Ser. No. 19/731,424, filed on Jul. 6, 2026 which is a continuation-in-part of U.S. patent application Ser. No. 17/922,729, filed on Nov. 1, 2022, which is a U.S. National Phase Application under 35 U.S.C. § 371 of International Patent Application No. PCT/IB2021/053658, filed May 1, 2021, which claims the benefit of Indian Patent Application number 202021018714, filed on May 1, 2020, Indian Patent Application number 202021018731, filed on May 1, 2020, and Indian Patent Application number 202021018735, filed on May 1, 2020, the contents of each of which are hereby incorporated by reference in their entirety.

### FIELD OF THE INVENTION

The present invention relates to a drug substance composition comprising omalizumab or a biosimilar of omalizumab having light chain and heavy chain, size variants including High molecular weight impurities (HMW) and Light molecular weight impurities (LMW), charge variants including acidic and basic variants in a suitable amount. In particular, the drug substance of the invention is stable at stress conditions for suitable period of time.

### BACKGROUND OF THE INVENTION

Omalizumab is a recombinant humanized monoclonal antibody directed against immunoglobulin E (IgE) and is widely used for the treatment of allergic asthma, chronic spontaneous urticaria, and other IgE-mediated disorders. Due to its complex molecular structure, post-translational modifications, and sensitivity to manufacturing conditions, omalizumab exhibits inherent molecular heterogeneity.

In the production of Omalizumab, which is produced in suspension cell culture using a Chinese Hamster Ovary (CHO) cell line, different kind of impurities often contain unwanted components, such as size variants or charge variants. Consequently, regulatory authorities require biosimilar drug substances to demonstrate consistent control of defined amount of impurities in antibody and long-term stability comparable to the reference biologic product. Despite advances in biosimilar manufacturing, there remains a need for pharmaceutical drug substances comprising biosimilar of omalizumab that exhibit high overall purity, a suitable light and heavy chain amount profile, High molecular weight impurities (HMW) and Light molecular weight impurities (LMW) profile, acidic and basic variants profile and their long-term stability distribution.

Additionally, the applicant's own earlier disclosure neither teaches nor suggests a drug substance composition having a substantially pure monomer of omalizumab or biosimilar of omalizumab, heavy chain and light chain in a suitable amount along with reduced impurities and enhanced stability when stored at 2 °C to about 8 °C for a suitable period. It would not have been expected by a person skilled in the art that such drug substance's stability for a suitable duration could be achieved within the presently claimed ranges. Drug substance preparation involves additional scientific processing steps after chromatographic elution and therefore earlier disclosure does not provide a reasonable expectation of success regarding drug substance stability.

Accordingly, there exists a need for improved pharmaceutical drug substance compositions of biosimilar of omalizumab that provide robust control of light chain and heavy chain amount, High molecular weight impurities (HMW) and Light molecular weight impurities (LMW) profile, acidic and basic variants profile that are amenable to scalable manufacturing processes, and demonstrate consistent long-term stability, thereby meeting regulatory expectations for biosimilarity, quality and shelf life.

### SUMMARY OF THE INVENTION

In an embodiment, the present invention discloses a drug substance composition comprising:
a monomer of an omalizumab or a biosimilar of an omalizumab comprising a light chain and a heavy chain in a suitable amount; and
one or more pharmaceutically acceptable excipients;
wherein, the suitable amount of the light chain and the heavy chain comprising;
a) the light chain is present in an amount of not less than 26% as determined by a reduced capillary electrophoresis sodium dodecyl sulfate (rCE-SDS); and
b) the heavy chain is present in an amount of not less than 63% as determined by a reduced capillary electrophoresis sodium dodecyl sulfate (rCE-SDS);
wherein, the monomer of omalizumab or biosimilar of omalizumab has a purity of at least more than 90% as determined by reduced size exclusion chromatography (rSEC);
wherein, the drug substance is stable and maintains the suitable amount of the heavy chain and the light chain upon storage at about 2 °C to about 8 °C for a suitable period;
wherein, the suitable period is selected from three days, seven days, fifteen days and thirty days.

In an embodiment, the present invention discloses the drug substance composition comprising:
a monomer of an omalizumab or a biosimilar of an omalizumab comprising a light chain and
a heavy chain in a suitable amount; and
one or more impurities;
wherein, the suitable amount of the light chain and the heavy chain comprising;
a) the light chain is present in an amount of not less than 26% as determined by a reduced capillary electrophoresis sodium dodecyl sulfate (rCE-SDS); and
b) the heavy chain is present in an amount of not less than 63% as determined by a reduced capillary electrophoresis sodium dodecyl sulfate (rCE-SDS);
wherein, the monomer of omalizumab or biosimilar of omalizumab has a purity of at least more than 90% as determined by reduced size exclusion chromatography (rSEC);
wherein, the drug substance is stable and maintains the suitable amount of the heavy chain and the light chain upon storage at about 2 °C to about 8 °C for a suitable period;
wherein, the suitable period is selected from three days, seven days, fifteen days and thirty days.

In an embodiment, the present invention discloses the drug substance composition consisting of:
a monomer of an omalizumab or a biosimilar of an omalizumab comprising a light chain and
a heavy chain in a suitable amount; and
one or more impurities;
wherein, the suitable amount of the light chain and the heavy chain comprising;
a) the light chain is present in an amount of not less than 26% as determined by a reduced capillary electrophoresis sodium dodecyl sulfate (rCE-SDS); and
b) the heavy chain is present in an amount of not less than 63% as determined by a reduced capillary electrophoresis sodium dodecyl sulfate (rCE-SDS);
wherein, the monomer of omalizumab or biosimilar of omalizumab has a purity of at least more than 90% as determined by reduced size exclusion chromatography (SEC);
wherein, the drug substance is stable and maintains the suitable amount of the heavy chain and the light chain upon storage at about 2 °C to about 8 °C for a suitable period;
wherein, the suitable period is selected from three days, seven days, fifteen days and thirty days.

In an embodiment, the present invention discloses the drug substance comprising:
a monomer of an omalizumab or a biosimilar of an omalizumab comprising a light chain and
a heavy chain in a suitable amount;
wherein, the suitable amount of the light chain and the heavy chain comprising:
a) the light chain is present in an amount of not less than 26% as determined by a reduced capillary electrophoresis sodium dodecyl sulfate (rCE-SDS); and
b) the heavy chain is present in an amount of not less than 63% as determined by a reduced capillary electrophoresis sodium dodecyl sulfate (rCE-SDS);
wherein, the monomer of omalizumab or biosimilar of omalizumab has purity of at least more than 90% as determined by reduced size exclusion chromatography (SEC);
wherein, the drug substance is stable and maintains the suitable amount of the heavy chain and the light chain upon storage at about 2 °C to about 8 °C for a suitable period;
wherein, the suitable period is selected from three days, seven days, fifteen days and thirty days. In a preferred embodiment, the drug substance composition has the pH in range of about 5.5 to about 7.

In one embodiment the pH of the drug substance composition is about 6.

In an embodiment, the excipients are selected from buffer and amino acid, wherein the buffer is selected from histidine, phosphate and acetate. In an embodiment, the buffer is phosphate buffer.

In an embodiment, the amino acid is selected from arginine, lysine, glycine and proline. In an embodiment, the amino acid is arginine.

In an embodiment, the one or more impurities are selected from high molecular weight impurities (HMW), low molecular weight impurities (LMW), acidic and basic variants.

In an embodiment, the light chain is present in an amount of about 26%, about 27%, about 28%, about 29%, about 30%, about 31%, about 32%, about 33%, about 34% and about 35%.

In an embodiment, the heavy chain is present in an amount of about 63%, about 64%, about 65%, about 66%, about 67%, about 68%, about 69%, about 70%, about 71%, about 72%, about 73%, about 74% and about 75%.

In an embodiment, the composition maintains stability in at least four batches.

In an embodiment, the batches are performed at scale of 50 L, 200 L, 500 L, 1000 L, 2000 L, 5000 L.

In an embodiment, the monomer of omalizumab or biosimilar of omalizumab is about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8% and about 99.9%, measured by reduced size exclusion chromatography (SEC).

In an embodiment, the high molecular weight impurities (HMW) are present in an amount of about 0.95%, about 0.9%, about 0.85%, about 0.80%, about 0.75%, about 0.70%, about 0.65%, about 0.60%, about 0.55%, about 0.50%, about 0.45%, about 0.40%, about 0.35%, about 0.30%, about 0.25%, about 0.21%, about 0.20%, about 0.18%, about 0.16%, about 0.15%, or about 0.1%.

In an embodiment, the low molecular weight impurities (LMW) are present in an amount of about 5%, about 4%, about 3%, about 2.9%, about 2.8%, about 2.7%, about 2.6%, about 2.5%, about 2.4%, about 2.3%, about 2.2%, about 2.1%, about 2.0%, about 1.9%, about 1.8%, about 1.7%, about 1.6%, about 1.5%, about 1.4%, about 1.3%, about 1.2%, about 1.1%, about 1.0%, about 0.9%, about 0.8%, about 0.7%, about 0.6%, or about 0.5%.

In an embodiment,
the acidic variants are present in an amount of below 11%;
the acidic variants are present in an amount of selected from 10.2%, 10%, 9.9%, 9.8%, 9.5%, 9.2%;
the acidic variants are present in an amount of below 9%;
the acidic variants are present in an amount of below about 8%;
the acidic variants are present in an amount of below about 7%; and
the acidic variants are present in an amount of below about 6%.

In an embodiment,
the basic variants are present in an amount of below 9%;
the basic variants are present in an amount of below about 8%;
the basic variants are present in an amount of selected from 7%, 6.6%, 6.2%, 6.1%;
the basic variants are present in an amount of below about 6%;
the basic variants are present in an amount of below about 5%; and
the basic variants are present in an amount of below about 4%.

### DETAILED DESCRIPTION OF THE INVENTION

The present invention provides a purification process which is capable to reduce various types of product and process related impurities.

In certain embodiment, the invention provides the reduction of few product related impurity by using CHT chromatography.

In certain embodiment, the invention provides the reduction of few product related impurity by using anion exchange chromatography.

In certain embodiment, the invention provides the reduction of product related impurity by using CHT and anion exchange chromatography.

In certain embodiment, the invention provides the reduction of product related impurity by using Affinity Chromatography, CHT and Anion Exchange Chromatography.

The term “antibody” includes an immunoglobulin molecule comprised of four polypeptide chains, two heavy (H) chains and two light (L) chains inter-connected by disulphide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. Omalizumab (Xolair®) is a recombinant DNA-derived humanized IgGlK monoclonal antibody that selectively binds to human immunoglobulin (IgE). The antibody has a molecular weight of approximately 149 kD. Xolair® is produced by a Chinese hamster ovary cell suspension culture in a nutrient medium containing the antibiotic gentamicin. Gentamicin is not detectable in the final product. Xolair® is a sterile, white, preservative-free, lyophilized powder contained in a single-use vial that is reconstituted with Sterile Water for Injection (SWFI), USP, and administered as a subcutaneous (SC) injection. The pI of the Omalizumab is less than 8, preferably about 7.6.

The term “acidic variant” or “acidic species” and “AV” used herein refer to the variants of a protein, e.g., an antibody or antigen-binding portion thereof, which are characterized by an overall acidic charge. For example, in monoclonal antibody (mAb) preparations, such acidic species can be detected by various methods, such as ion exchange, for example, WCX-10 HPLC (a weak cation exchange chromatography), or IEF (isoelectric focusing). Acidic variants of antibodies are formed through Chemical and enzymatic modifications such as deamidation and sialylation, respectively, result in an increase in the net negative charge on the antibodies and cause a decrease in p/values, thereby leading to formation of acidic variants. C-terminal lysine cleavage results in the loss of net positive charge and leads to acidic variant formation.

Another mechanism for generating acidic variants is the formation of various types of covalent adducts, e.g., glycation, where glucose or lactose can react with the primary amine of a lysine residue during manufacturing in glucose-rich culture media or during storage if a reducing sugar is present in the formulation.

The term “acidic variant” does not include process-related impurities. The term “process-related impurity,” as used herein, refers to impurities that are present in a composition comprising a protein but are not derived from the protein itself. Process-related impurities include, but are not limited to, host cell proteins (HCPs), host cell nucleic acids, chromatographic materials, and media components.

The term “anion exchange chromatography” or “anion exchange column” or “AEX” used herein is a form of ion exchange chromatography (IEX), which is used to separate molecules based on their net surface charge. Anion exchange chromatography, more specifically, uses a positively charged ion exchange resin with an affinity for molecules having net negative surface charges. Anion exchange chromatography is used both for preparative and analytical purposes and can separate a large range of molecules, from amino acids and nucleotides to large proteins. Here, we focus on the preparative anion exchange chromatography of proteins. The term “POROS 50 HQ” used herein is a Thermo Scientific™ POROS™ Strong Anion Exchange Resins (POROS AEX resins) are designed for charge-based chromatographic separation of biomolecules including recombinant proteins, monoclonal antibodies. Thermo Scientific™ POROS™ 50 HQ resin is functionalized with quaternized polyethyleneimine groups.

When “strong anion exchange” is used in flow through process the equation changes, the impurities are differentiated from the protein of interest, i.e., strong anion exchange is generally known for removal of protein A contaminant, HCP, DNA or virus in antibody purification. In a flow-through protocol, the sample and equilibration buffer are adjusted to conditions where contaminant molecules will still bind to the resin, but the protein of interest will not (because of the charge). This is achieved by increasing the salt concentration and/or increasing the pH of the buffers to a point below the pI of your molecule of interest.

As used herein the term “flow-through mode” or “flow-through” refers to purification process wherein antibody of interest does not bind to chromatography resin. In certain embodiment the at least 50% antibody of interest does not bind to chromatographic resin. In certain embodiment the at least 60% or 70% or 80% antibody of interest does not bind to chromatographic resin. However, process and product related impurities bind the chromatographic resin. In certain embodiment the at least 50% process and product related impurities bind to chromatographic resin. In certain embodiment the at least 60% or 70% or 80% process and product related impurities bind to chromatographic resin.

As used herein the term “column” or “resin” or “chromatographic resin or chromatographic column” are interchangeable.

The term “CHT” or “ceramic hydroxyapatite chromatography” is a form of calcium phosphate used in the chromatographic separation of biomolecules. Sets of five calcium doublets (C-sites) and pairs of —OH containing phosphate triplets (P-sites) are arranged in a repeating geometric pattern. Repeating hexagonal structures can be seen in electron micrographs of the material. Space-filling models and repeat structure from Raman spectroscopy have also been constructed. Hydroxyapatite has unique separation properties and unparalleled selectivity and resolution. It often separates proteins shown to be homogeneous by electrophoretic and other chromatographic techniques.

Applications of hydroxyapatite chromatography include the purification of different subclasses of monoclonal and polyclonal antibodies, antibodies that differ in light chain composition, antibody fragments, isozymes, supercoiled DNA from linear duplexes, and single-stranded from double stranded DNA. CHT ceramic hydroxyapatite is a spherical, macroporous form of hydroxyapatite. It has been sintered at high temperatures to modify it from a crystalline to a ceramic form.

The term “reduced Size Exclusion Chromatography” (rSEC) used herein refers to an analytical technique used primarily for monoclonal antibodies (mAbs) and proteins. It involves chemically reducing disulfide bonds (using agents like DTT) to separate heavy and light chains, followed by separation based on size and direct measurement of their molecular weights using Mass Spectrometry (MS).

The term “substantially pure antibody” used herein includes an antibody that is substantially free of impurity selected from product or process related impurity. In certain embodiment antibody is free of acidic variant, basic variant, low molecular weight and high molecular weight, substantially pure antibody has purity less than about 99% or less than about 98% or less than about 97% or less than about 95% or less than about 92% or less than about 90% or less than about 88% or less than about 85% or less than about 82% less than about 80% or less than about 75% or less than about 70% or less than about 65% or less than about 60% or less than 50%.

The term used “Size variants” refers to LMW, HMW, or aggregates.

The term used “low molecular weight” or “LMW” species or “LMW aggregates” which is a protein backbone-truncated fragments & considered as product-related impurities that contribute to the size heterogeneity of antibody. LMW species often have low or substantially reduced activity relative to the monomeric (i.e., main peak) form of the antibody and can lead to immunogenicity or potentially impact pharmacokinetic properties in vivo. As a result, LMW species are considered critical quality attributes that are routinely monitored during drug development and as part of release testing of purified drug product during manufacturing. The LMW has molecular weight selected from 23 kDa, 24 kDa, 25 kDa, 26 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 105 kDa, 110 kDa, 115 kDa, 120 kDa, 125 kDa, 126 kDa, and 127 kDa. In certain embodiment the LMW is selected from LC, HC, HH, and 2HL1.

The term “Light Chain” or “LC” of an antibody having a weight of 25 kDa is light or L chain. A typical antibody contains two light chain, lambda (λ) and kappa (κ). An antibody either has κ chains or λ chains, never one of each. There is no functional difference has been found between antibodies having λ or κ light chains, and either type of light chain can be found in antibodies of any of the five major classes. The ratio of the two types of light chain varies from species to species. However, sometime protein mixture comprises only one LC which is considered LMW impurity.

The term “Heavy Chain” or “HC” of an antibody having a weight of 50 kDa each is a heavy or H chain. There are several different types of heavy chain that define the class or isotype of an antibody. These heavy chain types vary between different animals. The heavy chains contain a series of immunoglobulin domains with one variable domain (VH) for binding an antigen and constant domains (CH1, CH2, etc.). The heavy chains with α and γ have approximately 450 amino acids and the heavy chains with μ and ε have approximately 550 amino acids. However, sometime protein mixture comprises only one HC which is considered LMW impurity.

The term “Two Heavy One Light Chain” or “2H1L” or “H2L” in antibody species possess a single light chain. The 2H1L species of antibody forms due to the β-elimination of a heavy chain cysteine residue, forming the inter-heavy and light chain disulfide bond, that leads to the formation of H2L species.

The term used “high molecular weight” or “HMW species” or “HMW aggregates” is product-related impurities that contribute to the size heterogeneity of antibody products. The formation of HMW species within a therapeutic antibody drug product as a result of protein aggregation can potentially compromise both drug efficacy and safety (e.g., eliciting unwanted immunogenic response). HMW is considered critical quality attribute that are routinely monitored during drug development and as part of release testing of purified drug product during manufacturing. In certain embodiment the HMW relates to aggregates.

The term used “aggregates” are classified based on types of interactions and solubility. Soluble aggregates are invisible particles and cannot be removed with a filter. Insoluble aggregates can be removed by filtration and are often visible to the human eye. Both types of aggregates cause problems in biopharma development. Covalent aggregates arise from the formation of a covalent bond between multiple monomers (i.e., main peaks) of a given peptide. Disulfide bond formation of free thiols is a common mechanism for covalent aggregation. Oxidation of tyrosine residues can lead to formation of bityrosine which often results in aggregation. Reversible protein aggregation typically results from weaker protein interactions they include dimers, trimers, multimers, among others.

The term used “basic variant” refers to variants can result from the presence of C-terminal lysine or glycine amidation, succinimide formation, amino acid oxidation or removal of sialic acid, which introduce additional positive charges or removal of negative charges; both types of modifications cause an increase in p/values.

The term used “high salt buffer” refers to high strength or high molality buffer.

The term used “Buffer A” and “Buffer B” is interchangeable with “first buffer” or “second buffer” respectively in CHT chromatography.

In another embodiment the invention provides a basic variant of Omalizumab selected from less than about 10% at least in five batches of 500 L scale wherein the basic variant is analyzed by CEX-HPLC. In an embodiment the invention provides a basic variants less than less than about 10%, about 9%, about 8%, about 7%, about 6%, about 5%, about 4%, about 3%, about 2%, and about 1% are reproducible and consistent in all the batches at 500 L.

As used herein the term “product-related impurity” refers to the impurity derived from the product of interest for example Basic variant or LMW.

As used herein the term “Analytical HPLC” refers to cation exchange chromatography (CEX-HPLC) and size exclusion chromatography (SE-HPLC).

The analytical HPLC technique CEX-HPLC provides separation based on the charge-to-size ratio under an electric field and is thus used to determine the charge variants comprising basic variants, basic variants and the desired protein. Among the different peaks obtained in the CEX-HPLC chromatogram, “the major peak” or “the main peak” represents the “desired protein” and the other peaks represent the basic charge variants and the basic charge variants. As used with CEX-HPLC, the term “desired protein” refers to a single, intact antibody molecule which has not undergone additional modifications like deamidation, oxidation, isomerization, clipping of C-terminal Lysine or variations in glycosylation leading to net change in the overall charge of the molecule. The area of the main peak or the major peak provides the amount of the desired protein present in the sample, similarly the area of the peaks representing the basic charge variants and the basic charge variants provides the amount of the “acidic” and the “basic” charge variants respectively present in the sample. The percentage of the area of the main peak or the major peak out of the total area of all the peaks represents the purity of the desired protein in the sample. Similarly the acidic and the basic charge variants percentage is determined. The ratio of basic charge variants to the main peak is obtained by dividing the percentage of basic charge variants by percentage of main peak. Similarly, the average value of the ratio of basic charge variants to the main peak for at least 2 batches at 500 L scale is referred to as the “average ratio of basic charge variants to the main peak”.

The analytical HPLC technique SE-HPLC provides separation based on the size and is thus used to determine the size variants comprising low molecular weight species, high molecular weight species and the desired protein or the main peak. Among the different peaks obtained in the SE-HPLC chromatogram, “the major peak” or “the main peak” represents the desired protein or the main peak and the other peaks represent the low molecular weight species and the high molecular weight species. As used with SE-HPLC, the term “desired protein” or “main peak” refers to a single, intact antibody molecule or the desired form of antibody molecule, representing the functional single antibody units. The area of the main peak or the major peak provides the amount of the desired protein or the main peak present in the sample, similarly the area of the peaks representing the low molecular weight species and the high molecular weight species provides the amount of the “low” and the “high” molecular weight species respectively present in the sample. The percentage of the area of the main peak or the major peak out of the total area of all the peaks represents the purity of the desired protein or the main peak in the sample. Similarly the low molecular weight species an the high molecular weight species percentage is determined. The ratio of low molecular weight species to the main peak or main peak is obtained by dividing the percentage of low molecular weight species by percentage of main peak or main peak. Similarly, the average value of the ratio of low molecular weight species to the main peak or main peak for at least 2 batches at large scale is referred to as the “average ratio of low molecular weight species to the main peak or main peak”.

The present invention surprisingly found the removal of basic variants and LMW through a strong anion exchange column by performing the column in a flow-through mode wherein the buffer solution pH is 7.0 to 7.3 marginally below the pI of the omalizumab.

The present invention provides the purified antibody composition obtained from strong anion exchange wherein the basic variants are less than 10% preferably less than 5% and LMW less than 2% preferably less than 0.5% which is under the acceptable limit of regulatory bodies. The present invention is very useful in reducing the burden in downstream processing by avoiding the use of multiple columns. In certain embodiment, the present invention avoids the use of HIC, and multimodal chromatography.

In another embodiment the invention provides a purity of Omalizumab selected from at least 90%, 95% and 98% at least in five batches of large scale wherein the purity is determined by SE-HPLC. In another embodiment the invention provides a purity of Omalizumab selected from at least about 70%, about 72%, about 74%, about 76%, about 78%, about 80%, about 82%, about 84%, about 86%, about 88%, and about 90% at least in five batches of large scale wherein the purity is determined by CEX-HPLC In an embodiment the invention provides a purity is reproducible and consistent in all the batches at large scale.

The term “consistency” or “consistent results” as used herein refers to the ability of the process to produce the same results or outcomes over multiple runs under the same conditions. It indicates the method's reliability and uniformity in performance, ensuring that repeated analyses yield similar data. Consistency or consistent results refer to the improvement in the parameters observed by following the process as described herein; for example, the percentage reduction observed in low molecular weight species by performing the CHT step (CHT load to CHT OP) as described herein provides consistent results in 5 batches.

The term “reproducibility” or “reproducible” as used herein refers to the ability of an analytical method to yield similar results when performed by different operators, in different laboratories, or using different equipment, under the same conditions. Consistency is crucial for validating the dependability and stability of the method whereas reproducibility is essential for validating the reliability and accuracy of data across different setting.

As used herein the term “column” or “resin” or “chromatographic resin or chromatographic column” are interchangeable.

The term “comprises” or “comprising” is used in the present description, it does not exclude other elements or steps. For the purpose of the present invention, the term “consisting of” is considered to be an optional embodiment, of the term “comprising of”. If hereinafter a group is defined to comprise at least a certain number of embodiments, this is also to be understood to disclose a group which optionally consists only of these embodiments.

As used throughout the specification and in the appended claims, the singular forms “a,” “an,” and “the” include the plural reference unless the context clearly dictates otherwise.

The term “about”, as used herein, is intended to refer to ranges of approximately 5-20% greater than or less than the referenced value. In certain circumstances, one of skill in the art will recognize that, due to the nature of the referenced value, the term “about” can mean more or less than a 5-20% deviation from that value. In some embodiments, the term “about” is intended to refer to ranges of approximately 5-10% greater than or less than the referenced value. The term “CE-SDS” is used herein to analyze LMW is selected from LC, HC, HH, and 2HL1. The term “analytical HPLC” comprises CEX HPLC used for the estimation of charge variants and SE-HPLC for the estimation of HMWs and LMWs.

In an embodiment, the “drug substance”, or “drug substance composition” and “composition” used herein are interchangeable.

The term “composition” used herein refers to a purified composition which is obtained from at least one column purification from downstream processing.

The term “five manufacturing batches” or ‘five batches’, or “four batches” or “four manufacturing batches” refers to the composition derived from 5 batches or 4 manufacturing batches of omalizumab or biosimilar of omalizumab.

The term ‘drug substance’ used herein refers to the active ingredient or a therapeutic molecule (e.g., monoclonal antibody, protein, vaccine) produced by living cells or microorganisms. It acts as the “active pharmaceutical ingredient” (API) before being formulated with excipients into the final drug product. The drug substance in one embodiment is free of surfactant, or a chelating agent.

The term ‘monomer’ used herein refers to monomer of the antibody or protein of interest percentage by Size Exclusion Chromatography (SEC) which is determined by comparing the area under the monomer peak to the total area of all peaks (aggregates, monomers, and fragments) in the chromatogram. It measures sample purity and structural integrity.

The term “substantially pure” used herein includes an antibody that is substantially free of impurity selected from product or process related impurity. In certain embodiment antibody is free of acidic variant, basic variant, low molecular weight and high molecular weight, substantially pure antibody has purity less than about 99% or less than about 98% or less than about 97% or less than about 95% or less than about 92% or less than about 90% or less than about 88% or less than about 85% or less than about 82% less than about 80% or less than about 75% or less than about 70% or less than about 65% or less than about 60% or less than 50%.

The term ‘stability’ or ‘stable’ used herein refers to the physical or chemical parameters of the drug substance maintained at 2 °C to about 8 °C for at least up to thirty days. The physical or chemical parameters includes monomer content, light chain and heavy chain.

In an embodiment ‘biosimilar of omalizumab’ is a highly similar biological medication to the omalizumab, which is a monoclonal antibody that targets Immunoglobulin E (IgE) to treat allergic asthma, chronic hives, and food allergies.

In an aspect, the term ‘oxidated variant’ used herein is a kind of basic impurity which typically introduces a +16 Da mass shift (e.g., methionine sulfoxide). Oxidated variants in monoclonal antibodies (mAbs) are produced primarily through the chemical degradation of specific amino acid residues, methionine (Met) and tryptophan (Trp) via Reactive Oxygen Species (ROS). These modifications typically happen during cell culture production, purification, or storage, often resulting in increased polarity, reduced stability, and potential loss of binding affinity. In an aspect, the ‘deamidated variant’ used herein refers to a modified protein where one or more asparagine (Asn) or glutamine (Gln) residues have been converted to aspartate (Asp) or glutamate (Glu), respectively, through a non-enzymatic process; wherein in deamidated variant, the deamidation introduces a negative charge where a neutral amide group previously existed.

The term ‘glycated variant’ used herein refers to deamidated with glycation, wherein the glucose or lactose can react with the primary amine of a lysine residue during manufacturing in glucose-rich culture media or during storage if a reducing sugar is present in the formulation.

The term ‘sialylated variant’ herein refers to a molecule, typically a glycoprotein or glycolipid, that has been modified by the addition of sialic acid, a sugar molecule, to its structure. Sialylation is a type of glycosylation where sialic acid is covalently attached to the terminal ends of glycan chains. These sialic acid residues play a crucial role in various biological processes, including cell-cell interactions, immune responses, and signalling. The terms ‘sialylated variant’ and ‘sialylated glycans’ are used interchangeably.

The term ‘succinimide variant’ herein refers to a modified form of succinimide, a cyclic imide with the chemical formula C4H5NO2. These variants are often studied for their potential biological activities and are found as intermediates or modifications in proteins. Specifically, a succinimide variant of a protein can arise from the cyclization of an aspartic acid or asparagine residue, forming a cyclic imide ring. It also refers to Succinimide (SNN), an intermediate formed during asparaginyl deamidation or aspartyl dehydration in proteins, is generally hydrolysis-prone, leading to isomerization to L/D α/β-aspartyl residue, with the latter being considered deleterious to protein structure and function. An unusually stable SNN-mediated conformational rigidity through restriction of the backbone dihedral angle, ψ, enhances the thermostability of glutamine amidotransferase (GATase) from Methanocaldococcus jannaschii (Mj). Although several structural features involved in maintaining a stable SNN and imparting SNN-mediated thermostability have been identified in MjGATase, the residues in the protein that catalyse the rapid and complete conversion of Asn109 to SNN remain unknown. The investigation was done on several site-directed mutants of MjGATase for their ability to retain Asn109 side chain in the unmodified form. Mass spectrometric analysis of 10 single mutants enabled the identification of residues that impacted the proportion of SNN and Asn population in the protein sample which led to the generation of two double mutants that retained intact Asn109 side chain as observed in the mass spectra and crystal structures. These mutants with intact Asn residue at position 109, displayed lower thermal stability than the protein with the SNN modification and further understanding of the deprotonation mechanism was addressed using QM/MM MD metadynamics simulations

The ‘lysine variant’ used herein refers to the presence of incomplete cleavage of C-terminal lysine residues at the heavy chain of monoclonal antibodies (mAbs) during their production, leading to charge heterogeneity. While these variants don't typically impact antibody structure or function, they are important for ensuring product quality consistency and are often monitored in biopharmaceutical manufacturing.

The term ‘1× lysine variant’ or “K1’ refers to the lysine variant present at single chain of Omalizumab.

The term ‘2× lysine variant’ or ‘K2’ refers to the lysine variant present at double chain of Omalizumab.

The “Thermo Scientific™ ProPac™ Elite WCX” Weak Cation Exchange (WCX) liquid chromatography column used herein is designed for protein characterization including therapeutic proteins, monoclonal antibodies (mAbs), antibody-drug conjugates (ADCs) and biosimilar proteins, this column offers high resolution, highly efficient separation of proteins and their charge variants. The unique column chemistry has excellent performance over a wide range of pH, temperatures and mobile phase compositions with excellent recovery and low carryover.

In an aspect, the ‘1× lysine variant’ (K1) denotes one chain of C-terminal Lysine variant and ‘2× Lysine variant’ (K2) denotes two chains of C-terminal Lysine variant both; wherein 1×Lysine variant shows truncation at one chain and 2× lysine variant at 2 chains of C-terminal.

The term “Good Manufacturing Practice” or “GMP” refers to a regulatory framework defined by agencies such as the FDA, EMA, and WHO, which establishes standards for manufacturing processes to ensure identity, strength, quality, purity, and safety of pharmaceutical products. The term “buffer exchange” refers to the process of substituting the solution surrounding a protein with a different buffer system. This is typically achieved by diafiltration or repeated concentration and dilution cycles to prepare the protein for downstream purification, refolding, or formulation.

The term “excipients” or “pharmaceutically acceptable excipients” refers to non-active ingredients added during the formulation of the drug substance to improve stability, prevent aggregation, enhance solubility, or facilitate administration. Examples include disaccharides, surfactants, chelators, buffering agents, amongst others.

The term “Host cell proteins” or “HCP” used herein refers to the proteins other than the protein of interest generated by host organisms or cell lines pertaining to cellular metabolism during the production of the protein of interest considered as process-related protein impurities.

The term “Purified protein mixture” used herein refers to the solution comprising the fusion protein of interest and reduced one or more impurities as compared to the collected protein mixture.

The term “Product quality” used herein refers to the identity, purity, potency, stability, and safety of the protein of interest determined by the predefined product quality attributes for example glycan profile, charge variants profile, size variants profile, etc. which impacts the degree to which a drug substance or product meets its intended use and fulfils its inherent properties.

The term ‘Capillary Electrophoresis-Sodium Dodecyl Sulfate’ (CE-SDS) is an automated, high-throughput analytical technique used to separate and quantify proteins based on molecular weight, primarily replacing traditional SDS-PAGE in biopharmaceutical quality control. It provides high resolution for analyzing purity, monomeric content, and fragmentation of monoclonal antibodies (mAbs).

The term ‘non-reduced CE-SDS’ used herein operate under non-reducing condition (nrCE-SDS) which is used to monitor the purity of denatured intact antibodies while CE-SDS of reduced antibodies (rCE-SDS) is used to monitor intact LC, HC, and non-glycosylated HC. In non-reduced CE-SDS, the reducing agent is omitted. The entire protein structure is denatured only to unfold its 3D shape and mask it with a uniform negative charge. It is used to evaluate the purity of the intact, whole protein (e.g., an entire mAb structure), and to measure low-molecular-weight (LMW) fragments or high-molecular-weight (HMW) covalent aggregates. Alkylating agents are often used to prevent artificial disulfide shuffling during preparation. The term ‘reduced CE-SDS’ or “rCE-SDS” used herein refers to CE-SDS in which a reducing agent (such as DTT or (beta)-mercaptoethanol) is added to the sample in order to break the covalent disulfide bridges that link protein subunits together. It dissociates the intact molecule into its individual heavy chains (HC) and light chains (LC). It is used to quantify the amount of heavy and light chains, detect non-reducible size variants, and determine heavy chain glycosylation occupancy.

The ‘MES buffer’ used herein in the context of buffers typically stands for 2-(N-Morpholino) ethane sulfonic acid. It's a zwitterionic buffer commonly used in biological and biochemical experiments to maintain stable pH conditions. MES is a Good's buffer, known for its ability to resist changes in pH within a specific range.

The term ‘isoaspartate variant’ used herein refers to the Isoaspartate (isoAsp) which is a common non-enzymatic post-translational modification (PTM) that arises from the spontaneous deamidation of asparagine (Asn) or isomerization of aspartic acid (Asp) residues. It is a major form of protein degradation, often acting as a key impurity in therapeutic proteins like monoclonal antibodies (mAbs).

The term ‘isoimide variant’ used herein refers to isoimide (polyisoimide) which is an isomeric form of imide used primarily to enhance the processability and solubility of polyimides, often acting as a precursor that transforms into the imide form upon heating, while isoimide itself is not always considered a “basic” impurity in the pH sense, it acts as a structural impurity or variant in polyimide synthesis, often present at levels of 20% or more in certain deposition techniques.

The term ‘amidated variant’ used herein refers to a form of post-translational modification (PTM) where the C-terminal amino acid typically a proline on the heavy chain which is modified to contain an amide group instead of a free carboxyl group.

The term ‘UV detector’ used herein in HPLC measures the absorbance of ultraviolet light by analytes (typically 190-400 nm) to quantify compounds as they elute from the column. It operates using a deuterium lamp to pass light through a flow cell, applying the Beer-Lambert law to relate light absorption to concentration. Common types include single-wavelength, variable-wavelength, and diode-array detectors (DAD).

The term ‘Percent area’ used herein refers to a quantitative method determining a component's relative amount by dividing its peak area by the total area of all detected peaks and multiplying by 100.

The term ‘main peak purity’ used herein refers to a chromatographic analysis technique, commonly using diode-array detection (DAD) or mass spectrometry (MS) in HPLC, to verify if a single peak in a chromatogram consists of one pure substance rather than multiple co-eluting compounds. It assesses spectral homogeneity across the peak to confirm the absence of impurities.

In an embodiment, the term ‘main peak including K1/K2 peaks’ used herein refers to % area of main peak purity of biosimilar of omalizumab includes % area of 1× lysine variants (K1) and 2× lysine variant (K2).

In an embodiment, the term ‘basic charge variant excluding K1/K2 peaks’ used herein refers to % area of basic variant of biosimilar of omalizumab without or excluding % area of 1× lysine variants (K1) and 2× lysine variant (K2).

The term ‘buffer’ used herein is generally defined to prevent damage, absorbs shock, or resists sudden changes. The term has specific meanings across several different fields, primarily referring to a protective barrier, a chemical solution that resists pH changes, or a temporary computer memory space.

The term ‘Size Exclusion Chromatography’ (SEC) used herein is a separation technique that sorts molecules in solution by their size (hydrodynamic radius) rather than their chemical properties. It is widely used to purify proteins, separate biological complexes, and determine the molecular weight distribution of synthetic and natural polymers.

An embodiment of the present invention provides a drug substance composition of monoclonal antibody, preferably an anti-IgE antibody such as omalizumab or a biosimilar of omalizumab.

The present invention relates to a drug substance composition of omalizumab or a biosimilar thereof having a controlled structural integrity, impurity profile, and stability characteristics. In an embodiment, the invention provides a composition wherein the antibody is predominantly present in a monomeric form, while maintaining a defined distribution of heavy chain and light chain species as determined under reducing analytical conditions.

In an embodiment, the drug substance composition comprises an anti-IgE monoclonal antibody, preferably omalizumab or a biosimilar thereof, in a highly purified form. The antibody is characterized by a monomer content that exceeds conventional purity standards typically achievable in downstream processing, while simultaneously controlling fragmentation and aggregation pathways along with maintaining a suitable heavy chain to light chain ratio.

In certain embodiments, the drug substance composition exhibits a defined polypeptide composition wherein the light chain fraction and the heavy chain fraction are within the reproducible ranges when analyzed using reduced CE-SDS techniques.

In certain embodiments, the levels of high molecular weight species and low molecular weight species in the drug substance composition is maintained below levels typically observed in standard purification processes, or below detection limits.

In another embodiment, the drug substance composition demonstrates stability over a defined storage period when maintained at suitable temperatures. Stability, in this context, refers to the ability of the composition to retain its structural and purity attributes, including monomer content, impurity levels, and chain distribution.

In certain embodiments, the drug substance composition comprises one or more pharmaceutically acceptable excipients. Suitable excipients include buffering agents and amino acids that contribute to stabilization of the protein structure.

In an embodiment, the excipients are selected from buffers and amino acid, wherein the buffer is selected from histidine, phosphate and acetate. In an embodiment, the buffer is phosphate buffer.

In an embodiment, the amino acid is selected from arginine, lysine, glycine and proline. In an embodiment, the amino acid is arginine.

In an embodiment, the buffering agents are selected from but not limited to buffers such as phosphate, histidine, or acetate amongst others to maintain the pH within a narrow physiological range.

In another embodiment, the amino acids are selected from but not limited to arginine, lysine, glycine, proline amongst others where the amino acids such as arginine as a stabilizer by reducing aggregation or surface adsorption. The combination of the buffering agent and stabilizer amino acid contributes synergistically to maintain conformational stability and preventing degradation during storage.

In a preferred embodiment, the drug substance composition has the pH in range of about 5.5 to about 7. In one embodiment the pH of the drug substance composition is about 6.

In another embodiment, the composition of the present invention demonstrates reproducibility across multiple manufacturing batches. This reproducibility indicates that the process is robust and scalable, and that the quality attributes of the drug substance composition are not significantly affected by scale-up operations.

In an embodiment, the composition of the invention is reproduced in at least about four batches. In an embodiment, the composition of the invention is reproduced in at least about five batches. In an embodiment, the composition of the invention is reproduced in at least about six batches. In an embodiment, the composition of the invention is reproduced in at least about seven batches. In an embodiment, the composition of the invention is reproduced in at least about eight batches. In an embodiment, the composition of the invention is reproduced in at least about nine batches. In an embodiment, the composition of the invention is reproduced in at least about ten batches.

In certain embodiments, the multiple batches produced at varying scales exhibit comparable monomer purity, impurity distribution, and chain integrity, thereby confirming that the composition is suitable for large-scale manufacturing. Such consistency is critical for regulatory acceptance and ensures that the drug substance can be reliably produced with uniform quality.

In an embodiment, the drug substance comprising omalizumab or a biosimilar thereof is characterized by a controlled distribution of heavy chain and light chain species, wherein the relative proportion of the heavy chain to the light chain is maintained within a defined and reproducible range when analyzed under reducing conditions. In an embodiment, the drug substance comprising omalizumab or a biosimilar thereof is characterized by a defined distribution of heavy chain and light chain species, wherein the light chain is present within a range of about 26% to about 35% and the heavy chain is present within a corresponding range of about 63% to about 75%, as determined under reducing conditions.

In an embodiment, the light chain is present in an amount of about 26%, about 27%, about 28%, about 29%, about 30%, about 31%, about 32%, about 33%, about 34% and about 35%. In an embodiment, the heavy chain is present in an amount of about 63%, about 64%, about 65%, about 66%, about 67%, about 68%, about 69%, about 70%, about 71%, about 72%, about 73%, about 74% and about 75%.

In an embodiment, the drug substance comprises of a monomer of omalizumab or a biosimilar of omalizumab, and one or more impurities. The impurities are selected from but not limited to one or more product-related impurities selected from high molecular weight species, low molecular weight species, and charge variants including acidic and basic variants.

In certain embodiments, the high molecular weight species and low molecular weight species, are maintained at low levels relative to the total protein content. In an embodiment, the high molecular weight impurities are present in an amount less than about 1% as analyzed by size exclusion chromatography (SEC). In another embodiment, the low molecular weight impurities are present in an amount less than about 5% as analyzed by non-reduced capillary electrophoresis sodium dodecyl sulfate (CE-SDS). In an embodiment, the high molecular weight impurities (HMW) are present in an amount of about 0.9%, about 0.8%, about 0.7%, about 0.6%, about 0.5%, about 0.4%, about 0.35%, about 0.3%, about 0.21%, about 0.2%, about 0.16%, about 0.18%, about 0.1%. In an embodiment, the low molecular weight impurities (LMW) are present in an amount of about 5%, about 4%, about 3%, about 2.4%, about 2.2%, about 2%, about 1.5%, about 1.3%, about 1%, about 0.5%.

In another embodiment, the impurity profile comprises charge variants including acidic variants and basic variants. Such variants may include, without limitation, deamidation, glycation, sialylation, oxidation, incomplete processing, or lysine retention. The composition of the present invention is characterized by maintaining the levels of acidic and basic variants within defined and reproducible limits.

In an embodiment, the acidic variants are present in an amount of below 13%. In another embodiment, the basic variants are present in an amount of below 10%. In one embodiment, the acidic variants are present in an amount of below 11%. In another embodiment, the acidic variants are present in an amount of selected from 10.2%, 10%, 9.9%, 9.8%, 9.5%, 9.2%. In another embodiment, the acidic variants are present in an amount of below 9%, below about 8%, below about 7%, below about 6%. In another embodiment, the basic variants are present in an amount of below 9%, below about 8%, below about 6%, below about 5%, below about 4%. In another embodiment, the basic variants are present in an amount of selected from 7%, 6.6%, 6.2%, 6.1%. In another embodiment, acidic variants are selected from deamidated variants, glycated variants and sialylated variants amongst others and the total basic variants are selected from succinimide variants, 1× lysine variants (K1), 2× lysine variants (K2), isoaspartate variants, isoimide variants and amidated variants amongst others. The acidic variants and the basic variants are measured by high performance-cation exchange chromatography (HP-CEX) analysis by UV detector.

In another embodiment, the monomer of biosimilar of omalizumab in the drug substance is about 91%, about 92%, about 93%, about 94%, about 95%, about 96%, about 97%, about 98% and about 99%, about 99.1%, about 99.2%, about 99.3%, about 99.4%, about 99.5%, about 99.6%, about 99.7%, about 99.8%, about 99.9%, measured by reduced size exclusion chromatography (SEC).

In another embodiment, the drug substance composition exhibits reproducibility and consistency across multiple manufacturing batches produced at different scales, including but not limited to about 50 L, about 100 L, about 200 L, about 500 L, about 1000 L, about 2000 L and about 5000 L, without adversely affecting the structural and quality attributes of the antibody. In particular, batches produced across such scales exhibit comparable impurity profiles, heavy chain and light chain distribution, and monomer purity, thereby indicating robustness of the manufacturing process.

In an embodiment, the composition maintains stability in at least four batches. In an embodiment, the batches are performed at scale of 50 L, 200 L, 500 L, 1000 L, 2000 L, 5000 L. In an embodiment, the drug substance is stable under refrigerated conditions, particularly when stored at temperatures ranging from about 2 °C to about 8 °C. The composition retains its monomer content, impurity profile, and heavy chain/light chain distribution for a period of at least about fifteen days, at least about twenty days, at least about thirty days. The minimal variation observed in these attributes over time indicates that the composition is stable and capable of maintaining its quality characteristics during storage and handling.

The embodiment of the invention provides a drug substance composition with high monomer purity, controlled impurity levels, defined heavy chain and light chain distribution, and stability under refrigerated storage conditions are achieved simultaneously, resulting in a robust and consistent composition suitable for pharmaceutical use.

In an embodiment, the present invention discloses a drug substance composition comprising:
a monomer of an omalizumab or a biosimilar of an omalizumab comprising a light chain and
a heavy chain in a suitable amount; and
one or more pharmaceutically acceptable excipients;
wherein, the suitable amount of the light chain and the heavy chain comprising;
a) the light chain is present in an amount of not less than 26% as determined by a reduced capillary electrophoresis sodium dodecyl sulfate (rCE-SDS); and
b) the heavy chain is present in an amount of not less than 63% as determined by a reduced capillary electrophoresis sodium dodecyl sulfate (rCE-SDS);
wherein, the monomer of omalizumab or biosimilar of omalizumab has a purity of at least more than 90% as determined by reduced size exclusion chromatography (SEC);
wherein, the drug substance is stable and maintains the suitable amount of the heavy chain and the light chain upon storage at about 2 °C to about 8 °C for a suitable period;
wherein, the suitable period is selected from three days, seven days, fifteen days and thirty days.

In an embodiment, the present invention discloses the drug substance composition comprising:
a monomer of an omalizumab or a biosimilar of an omalizumab comprising a light chain and
a heavy chain in a suitable amount; and
one or more impurities;
wherein, the suitable amount of the light chain and the heavy chain comprising;
a) the light chain is present in an amount of not less than 26% as determined by a reduced capillary electrophoresis sodium dodecyl sulfate (rCE-SDS); and
b) the heavy chain is present in an amount of not less than 63% as determined by a reduced capillary electrophoresis sodium dodecyl sulfate (rCE-SDS);
wherein, the monomer of omalizumab or biosimilar of omalizumab has a purity of at least more than 90% as determined by reduced size exclusion chromatography (SEC);
wherein, the drug substance is stable and maintains the suitable amount of the heavy chain and the light chain upon storage at about 2 °C to about 8 °C for a suitable period wherein the suitable period is selected from three days, seven days, fifteen days and thirty days.

In an embodiment, the present invention discloses the drug substance composition consisting of:
a monomer of an omalizumab or a biosimilar of an omalizumab comprising a light chain and
a heavy chain in a suitable amount; and
one or more impurities;
wherein, the suitable amount of the light chain and the heavy chain comprising;
a) the light chain is present in an amount of not less than 26% as determined by a reduced capillary electrophoresis sodium dodecyl sulfate (rCE-SDS); and
b) the heavy chain is present in an amount of not less than 63% as determined by a reduced capillary electrophoresis sodium dodecyl sulfate (rCE-SDS);
wherein, the monomer of omalizumab or biosimilar of omalizumab has a purity of at least more than 90% as determined by reduced size exclusion chromatography (SEC);
wherein, the drug substance is stable and maintains the suitable amount of the heavy chain and the light chain upon storage at about 2 °C to about 8 °C for a suitable period;
wherein, the suitable period is selected from three days, seven days, fifteen days and thirty days.

In an embodiment, the present invention discloses drug substance comprising:
a monomer of an omalizumab or a biosimilar of an omalizumab comprising a light chain and
a heavy chain in a suitable amount;
wherein, the suitable amount of the light chain and the heavy chain comprising;
a) the light chain is present in an amount of not less than 26% as determined by a reduced capillary electrophoresis sodium dodecyl sulfate (rCE-SDS); and
b) the heavy chain is present in an amount of not less than 63% as determined by a reduced capillary electrophoresis sodium dodecyl sulfate (rCE-SDS);
wherein, the monomer of omalizumab or biosimilar of omalizumab has a purity of at least more than 90% as determined by reduced size exclusion chromatography (SEC);
wherein, the drug substance is stable and maintains the suitable amount of the heavy chain and the light chain upon storage at about 2 °C to about 8 °C for a suitable period;
wherein, the suitable period is selected from three days, seven days, fifteen days and thirty days.

### Examples

The present invention provides following examples for illustrative purpose and its scope should not be considered limited to the following examples.

### Example 1—Purification of Monoclonal Antibody by Performing Affinity Column Followed by Anion Exchange Column

All chromatographic processes were carried out using an AKTA Pure 150 system from GE Healthcare. Concentration of protein samples were determined by measuring absorbance at 280 nm using Shimadzu Spectrophotometer. Mabselect Sure LX resin media obtained from GE Healthcare. Vantage columns and C 10/20 columns were obtained from Millipore Corporation and GE Healthcare respectively. Turbidity measurements were measured using from Thermo scientific. All Chemicals were obtained from JTB or Merck Millipore and were of GMP grade. A monoclonal antibody molecule capable to bind to IgE molecule expressed in Chinese Hamster Ovary (CHO) cell line is captured using Protein A (Mab Select Sure LX, GE Healthcare) packed in VL 11/250 or C10/20 column. The residence time is 4 min for all the phases. After equilibration with Tris Acetate+100 mM NaCl, pH 6.8-7.2 clarified harvest is loaded at ≤45 mg/mL of the resin. After loading, the column is washed with equilibration buffer (wash 1) followed by wash 2 and wash 3 buffer as mentioned in Table 1. Affinity chromatography step is operated in bind and eluate mode and collection is done from 50 mAU (0.25 AU/cm) ascending to 50 mAU (0.25 AU/cm) descending of the peak. Protein A output is analysed with SE-HPLC, CEX-HPLC for size and charge variants. The experimental design for Protein A step is summarized in Table 1.

TABLE 1
Experimental design for Protein A

| | | Residence | Column |
|---|---|---|---|
| | | Time | Volume |
| Step | Buffer | (min) | (CV) |
| WFI | WFI | 4 | 2-3 |
| Sanitization | 0.1M NaOH | 4 | 2-3 |
| Equilibration | 20 mM Tris-acetate + | 4 | 5 |
| | 100 mM | | |
| | NaCl, pH 7.0 ± 0.2 | | |
| Load | Clarified Harvest | 4 | Till loading |
| | | | volume |
| Wash 1 | 20 mM Tris-acetate + | 4 | 3 or till |
| | 100 mM NaCl, | | conductivity |
| | pH 7.0 ± 0.2 | | achieved |
| Wash 2 | 20 mM Tris-acetate + | 4 | 3 or till |
| | 1M NaCl, pH 7.0 ± 0.2 | | conductivity |
| | | | achieved |
| Wash 3 | 20 mM Tris-acetate, | 4 | 5-6 |
| | pH 5.5 ± 0.2 | | |
| Elution | 200 mM Acetic acid, | 4 | 3 |
| | pH 3.0 ± 0.1 | | |
| Neutralization | 20 mM Tris-acetate + | 4 | 3 |
| wash | 100 mM NaCl, | | |
| | pH 7.0 ± 0.2 | | |
| Sanitization | 0.1M NaOH | 4 | 2-3 |
| Neutralization | 20 mM Tris-acetate + | 4 | 5 |
| wash | 100 mM NaCl, | | |
| | pH 7.0 ± 0.2 | | |
| Storage | 2% Benzyl alcohol in | 4 | 2-3 |
| | Wash 3 buffer | | |

Eluted protein is further subjected to viral inactivation and neutralization. After neutralization, protein has been filtered by 0.2 μm filter.

Post viral inactivation and neutralization step, the eluted protein mixture is further purified using Anion Exchange Chromatography resin (Poros HQ, Thermofisher) packed in C10/20 column.

The residence time is 4 min for all the phases. After equilibration with Tris Acetate, pH 7.0-7.3 Neutralized Protein A output is loaded at ≤100 mg/mL of the resin. The Load is diluted with water to meet the (mS/cm) conductivity specification i. e. ≤3.0 mS/cm before introducing it into the AEX column.

AEX step is operated in Flow-through (negative) mode and collection is done from 500 mAU ascending to 300 mAU descending of the peak. The column is washed using Tris Acetate, pH 7.0-7.3. AEX output is analysed with SE-HPLC, CEX-HPLC for size and charge variants. The experimental design for POROS 50 HQ step is summarized in Table 2.

TABLE 2
Experimental design for POROS 50 HQ (AEX)

| | | Residence | Column |
|---|---|---|---|
| | | Time | Volume |
| Step | Buffer | (min) | (CV) |
| Sanitization | 0.5N Sodium Hydroxide | 4 | 2-3 CV |
| Equilibration | 50 mM Tris Acetate, | 4 | 3-4 CV |
| | pH 7.2-7.3 | | |
| Load | 50 mM Tris Acetate, | 4 | Till loading |
| | pH 7.2-7.3 | | volume |
| Wash | 50 mM Tris Acetate, | 4 | Till absorbance |
| | pH 7.2-7.3 | | 1.5 AU/cm |
| Sanitization | 0.5N Sodium Hydroxide | 4 | 2-3 CV |
| Storage | 0.1N Sodium Hydroxide | 4 | 2-3 CV |

TABLE 3
SE (Size Exclusion)-HPLC (High performance Liquid
Chromatography Analysis)
SE-HPLC Analysis

| | Main Peak | |
|---|---|---|
| Sample | Purity (%) | HMW (%) |
| AEX IP | 94.62 | 4.71 |
| AEX OP | 99.34 | 0.07 |

Table 3 shows 99% purity of main peak and 98% reduction of HMW which is determined by SE-HPLC.

CEX-HPLC Analysis

| Sample | Main Peak + K1 + K2 (%) | Acidic Variants (%) |
|---|---|---|
| NPEL/AEX IP | 65.86 | 18.28 |
| AEX OP | 74.11 | 8.24 |

Table 4 shows 74% purity of main peak and approximately 55% reduction of acidic variants which is determined by CEX-HPLC.

### Example 2—Purification of Monoclonal Antibody by Performing Affinity Chromatography Followed by AEX Chromatography Followed by CHT Type 1 Resin

All chromatographic processes were carried out using an AKTA Pure 150 system from GE Healthcare. Concentration of protein samples were determined by measuring absorbance at 280 nm using Shimadzu Spectrophotometer. CHT Type I resin media obtained from Bio-rad. Vantage columns and C 10/20 columns were obtained from Millipore Corporation and GE Healthcare respectively. Turbidity measurements were measured using from Thermo scientific.

All Chemicals were obtained from JTB or Merck Millipore and were of GMP grade. A monoclonal antibody capable to bind to IgE expressed in Chinese Hamster Ovary (CHO) cell line is captured using Protein A (Mab Select Sure LX, GE Healthcare) packed in VL 11/250 column. Eluted protein is further subjected to viral inactivation and neutralization. After neutralization, protein has been filtered by 0.2 μm filter.

Post viral inactivation and neutralization step, the protein mixture is further purified using Anion Exchange Chromatography resin (Poros HQ, Thermofisher) packed in C10/20 column. Eluted protein from Anion Exchange Chromatography (AEX) is further polished using mixed-mode resin (CHT Type I, Bio-rad) packed in VL 11/250 column. The residence time is 4 min/3 min for all the phases. After equilibration with Na Phosphate, pH 6.8 Anion exchange (AEX) output is loaded at 12.5 mg/mL of the resin. The column is washed using 3 CV, Na Phosphate, pH 6.8. Bound protein of interest is eluted using linear gradient between 32 mM Na Phosphate and 400 mM Na Phosphate, pH 6.8. Eluted peak of interest is analysed with SE-HPLC, CEX-HPLC for size and charge variants. The experimental design and process for CHT Type I step is summarized in Table 5.

TABLE 5
Experimental design for CHT Type I

| | | Residence | Column |
|---|---|---|---|
| | | Time | Volume |
| Step | Buffer | (min) | (CV) |
| Sanitization | 0.5N Sodium | 4 | 3 CV and |
| | Hydroxide | | 0.5 hr hold |
| Charge | 400 mM Na | 4 | 3 CV |
| | Phosphate, pH 6.8 | | |
| Equilibration | 32 mM Na | 4 | 3 CV or conduc- |
| | Phosphate, pH 6.8 | | tivity end point |
| Load | AEX Eluate + 32 mM | 4 | Till loading |
| | Na Phosphate, pH 6.8 | | volume |
| Wash | 32 mM Na | 4 | 3 CV |
| | Phosphate, pH 6.8 | | |
| Gradient | Buffer A: Water | 3 to 4 | 19 CV |
| Elution | Buffer B: 400 mM | | |
| | Phosphate, pH 6.8 | | |
| | from 8 % to 22% B | | |
| Strip | 400 mM Phosphate, | 3 to 4 | 3 CV |
| | pH 6.8 | | |
| Sanitization | 0.5N Sodium | 3 to 4 | 3 CV and |
| | Hydroxide | | 0.5 hr hold |
| Storage | 0.1N Sodium | 3 to 4 | 3 CV |
| | Hydroxide | | |

### Example 3—Purification of Monoclonal Antibody by Performing Affinity Chromatography Followed by AEX Chromatography Followed by CHT XT Resin

All chromatographic processes were carried out using an AKTA Pure 150 system from GE Healthcare. Concentration of protein samples were determined by measuring absorbance at 280 nm using Shimadzu Spectrophotometer. CHT XT resin media obtained from Bio-rad. Vantage columns and C 10/20 columns were obtained from Millipore Corporation and GE Healthcare respectively. Turbidity measurements were measured using from Thermo scientific. All Chemicals were obtained from JTB or Merck Millipore and were of GMP grade. A monoclonal antibody capable to bind to IgE expressed in Chinese Hamster Ovary (CHO) cell line is captured using Protein A (Mab Select Sure LX, GE Healthcare) packed in VL 11/250 column.

Eluted protein is further subjected to viral inactivation and neutralization. After neutralization, protein has been filtered by 0.2 μm filter.

Post viral inactivation and neutralization step, the eluted protein mixture is further purified using Anion Exchange Chromatography resin (Poros HQ, Thermofisher) packed in C10/20 column.

Eluted protein from Anion Exchange Chromatography (AEX) is further polished using mixed-mode resin (CHT XT, Bio-rad) packed in VL 11/250 column. The residence time is 4 min/3 min for all the phases. After equilibration with Na Phosphate, pH 6.8 Anion exchange (AEX) output is loaded at 12.5 mg/mL of the resin. The column is washed using 3 CV, Na Phosphate, pH 6.8. Bound protein of interest is eluted using linear gradient between 40 mM Na Phosphate and 400 mM Na Phosphate, pH 6.8. Eluted peak of interest is analysed with SE-HPLC, CEX-HPLC for size and charge variants. The experimental design and process for CHT XT step is 5 summarized in Table 6-9.

TABLE 6
Experimental design for CHT XT

| | | Residence | Column |
|---|---|---|---|
| | | Time | Volume |
| Step | Buffer | (min) | (CV) |
| Sanitization | 0.5N Sodium | 4 | 3 CV and |
| | Hydroxide | | 0.5 hr hold |
| Charge | 400 mM Na | 4 | 3 CV |
| | Phosphate, pH 6.8 | | |
| Equilibration | 40 mM Na | 4 | 3 CV or conduc- |
| | Phosphate, pH 6.8 | | tivity end point |
| Load | AEX Eluate + 40 mM | 4 | Till loading |
| | Na Phosphate, pH 6.8 | | volume |
| Wash | 40 mM Na | 4 | 2-3 CV |
| | Phosphate, pH 6.8 | | |
| Gradient | Buffer A: Water | 3 to 4 | 19 CV |
| Elution | Buffer B: 400 mM | | |
| | Phosphate, pH 6.8 | | |
| | from 10% to 24% B | | |
| Strip | 400 mM Phosphate, | 3 to 4 | 3 CV |
| | pH 6.8 | | |
| Sanitization | 0.5N Sodium | 3 to 4 | 3 CV and |
| | Hydroxide | | 0.5 hr hold |
| Storage | 0.1N Sodium | 3 to 4 | 3 CV |
| | Hydroxide | | |

TABLE 7
SE-HPLC Analysis of Eluate of CHT Type I/XT Column

| Process output | Main Peak (%) | LMW (%) |
|---|---|---|
| NPEL | 94.55 | 0.84 |
| AEX OP | 99.31 | 0.67 |
| CHT Type I/XT OP | 99.99 | 0.00 |

It is evident from the data that composition obtained from CHT has substantially reduced approx. 100% LMW and purity of protein is 99.99% by SE-HPLC.

TABLE 8
Size related Variants: CE-SDS

| Process output | Total LMW (%) |
|---|---|
| NPEL | 8.91 |
| AEX OP | 7.48 |
| CHT Type I/XT OP | 1.42 |

It is evident from the data that composition obtained from CHT has substantially reduced approx. 81% LMW.

TABLE 9
Charge related Variants: CEX-HPLC

| Process output | Main Peak (%) | Basic (%) |
|---|---|---|
| NPEL | 73.38 | 12.83 |
| AEX OP | 77.33 | 12.19 |
| CHT Type I/XT OP | 84.72 | 5.27 |

Table 9 shows 84% purity of main peak and approximately 56% reduction of LMW which is determined by CEX-HPLC.

### Example 4—Purification of Monoclonal Antibody by Performing Affinity Column Followed by Anion Exchange Column (50 L Scale)

All chromatographic processes were carried out using an AKTA Pilot or AKTA Process from GE Healthcare. Concentration of protein samples were determined by measuring absorbance at 280 nm using Spectrophotometer. Mabselect Sure LX resin media obtained from GE Healthcare. Chromatographic 100/250 columns were obtained from Millipore Corporation or GE Healthcare or Repligen. All Chemicals used during manufacturing process were of GMP grade.

A monoclonal antibody molecule capable to bind to IgE molecule expressed in Chinese Hamster Ovary (CHO) cell line is captured using Protein A (Mab Select Sure LX, GE Healthcare) packed in Chromatographic 100/250 column. The residence time is 4 min for all the phases. After equilibration with Tris HCl+150 mM NaCl, pH 6.8-7.2 clarified harvest is loaded at ≤45 mg/mL of the resin. After loading column is washes with equilibration buffer (wash 1) followed by wash 2 and wash 3 buffer as mentioned in Table 10. Affinity chromatography step is operated in bind and eluate mode and collection is done from 0.25 AU/cm ascending to 0.25 AU/cm descending of the peak. Protein A output is analysed with SE-HPLC, CEX-HPLC for size and charge variants. The experimental design for Protein A step is summarized in Table 10.

TABLE 10
Experimental design for Protein A

| | | Residence | Column |
|---|---|---|---|
| | | Time | Volume |
| Step | Buffer | (min) | (CV) |
| Sanitization | 0.1M NaOH | 4 | 2-3 |
| Equilibration | 20 mM Tris-HCl + 150 mM | 4 | 5 or to pH |
| | NaCl, pH 7.0 ± 0.2 | | end point |
| Load | Clarified Harvest | 4 | Till loading |
| | | | volume |
| Wash 1 | 20 mM Tris-HCl + 150 mM | 4 | 3 |
| | NaCl, pH 7.0 ± 0.2 | | |
| Wash 2 | 20 mM Tris-HCl + 1M NaCl, | 4 | 5 |
| | pH 7.0 ± 0.2 | | |
| Wash 3 | 30 mM Sodium acetate, pH | 4 | 7 |
| | 5.5 ± 0.2 | | |
| Elution | 200 mM Sodium acetate, pH | 4 | 5 |
| | 3.5 ± 0.1 | | |
| Strip | 100 mM Acetic acid | 4 | 3 |
| Equilibration | 20 mM Tris-HCl + 150 mM | 4 | 3 |
| | NaCl, pH 7.0 ± 0.2 | | |
| Sanitization | 0.1M NaOH | 4 | 3 |
| Equilibration | 20 mM Tris-HCl + 150 mM | 4 | 5 or to pH |
| | NaCl, pH 7.0 ± 0.2 | | end point |
| Storage | 20% Ethanol | 4 | 3 |

Eluted protein is further purified using Anion Exchange Chromatography resin (POROS 50 HQ, Thermofisher) packed in Chromatographic 100/250 column. The residence time is 4 min for all the phases. After equilibration with Tris HCl, pH 7.1-7.3 Neutralized Protein A output is loaded at 70 to 145 mg/mL of the resin. The Load is diluted with water to meet the (mS/cm) conductivity specification i. e. ≤2.0 mS/cm before introducing it into the AEX column.

AEX step is operated in Flow-through (negative) mode and collection is done from 2.0 AU/cm ascending to 2.5 AU/cm descending of the peak. The column is washed using Tris HCl, pH 7.1-7.3. AEX output is analysed with SE-HPLC, CEX-HPLC for size and charge variants. The experimental design for POROS 50 HQ step is summarized in Table 11.

TABLE 11
Experimental design for AEX

| | | Residence | |
|---|---|---|---|
| | | Time | Column Volume |
| Step | Buffer | (min) | (CV) |
| Sanitization | 0.5N Sodium Hydroxide | 4 | 3 CV |
| Charge | 20 mM Tris HCl, 1M | 4 | 5 CV or pH end |
| | NaCl pH 7.2 | | point |
| Equilibration | 20 mM Tris HCl, pH 7.2 | 4 | 5 CV or pH end |
| | | | point |
| Load | AEX Load pH 7.2 | 4 | Till loading |
| | conductivity ≤ 2.0 | | volume |
| | mS/cm | | |
| Chase | 20 mM Tris HCl, pH 7.2 | 4 | 3 CV |
| Strip | 20 mM Tris HCl, 1M | 4 | 3 CV |
| | NaCl pH 7.2 | | |
| Sanitization | 0.5N Sodium Hydroxide | 4 | 3 CV |
| Storage | 0.1N Sodium Hydroxide | 4 | 3 CV |

TABLE 12
SE (Size Exclusion)-HPLC (High performance
Liquid Chromatography Analysis)
SE-HPLC Analysis

| Sample | Main Peak Purity (%) | HMW (%) |
|---|---|---|
| AEX IP | 95.52 | 3.80 |
| AEX OP | 99.45 | 0.14 |

Table 12 shows 99% purity of main peak and 96% reduction of HMW which is determined by SE-HPLC.

TABLE 13
shows the results of Cation Exchange-High performance Liquid
Chromatography comparing the main peak with acidic variants.
CEX-HPLC Analysis

| Sample | Main Peak (%) | Acidic Variants (%) |
|---|---|---|
| NPEL/AEX IP | 66.53 | 15.80 |
| AEX OP | 67.47 | 11.78 |

Table 13 shows 67% purity of main peak and approximately 25% reduction of acidic variants which is determined by CEX-HPLC.

### Example 5—Purification of Monoclonal Antibody by Performing Affinity Chromatography Followed by AEX Chromatography Followed by CHT XT Resin

All chromatographic processes were carried out using an AKTA Pilot or AKTA Process system from GE Healthcare. Concentration of protein samples were determined by measuring absorbance at 280 nm using Spectrophotometer. CHT XT resin media obtained from Bio-rad. Chromatographic 100/250 columns were obtained from Millipore Corporation or GE Healthcare or Repligen. All Chemicals used during manufacturing process were of GMP grade. A monoclonal antibody capable to bind to IgE expressed in Chinese Hamster Ovary (CHO) cell line is captured using Protein A (Mab Select Sure LX, GE Healthcare) packed in Chromatographic 100/250 column. Eluted protein is further subjected to viral inactivation and neutralization followed by depth filtration. After depth filtration, protein has been filtered by 0.2 μm filter.

Post depth filtration step, the protein mixture is further purified using Anion Exchange Chromatography resin (Poros HQ, Thermofisher) packed in Chromatographic 100/250 column.

Eluted protein from Anion Exchange Chromatography (AEX) is further polished using mixed-mode resin (CHT XT, Bio-rad) packed in Chromatographic 100/250 column. The residence time is 4 min/5 min for all the phases. After equilibration with Na Phosphate, pH 6.8 Anion exchange (AEX) output is loaded at maximum of 12.5 mg/mL of the resin. The column is washed using 3 CV, Na Phosphate, pH 6.8. Bound protein of interest is eluted using linear gradient between 40 mM Na Phosphate and 400 mM Na Phosphate, pH 6.8. Eluted peak of interest is analysed with SE-HPLC, CEX-HPLC for size and charge variants. The experimental design and process for CHT XT step is summarized in Table 14-17.

TABLE 14
Experimental design for CHT XT

| | | Residence | Column |
|---|---|---|---|
| | | Time | Volume |
| Step | Buffer | (min) | (CV) |
| Sanitization | 0.5N Sodium Hydroxide | 4 to 5 | 3 CV and |
| | | | 0.5 hr hold |
| Charge | 400 mM Na Phosphate, | 4 to 5 | 3 CV |
| | pH 6.8 | | |
| Equilibration | 40 mM Na Phosphate, | 4 to 5 | 3 CV or |
| | pH 6.8 | | conductivity |
| | | | end point |
| Load | AEX Eluate + 40 mM Na | 4 to 5 | Till loading |
| | Phosphate, pH 6.8 | | volume |
| Wash | 40 mM Na Phosphate, pH 6.8 | 4 to 5 | 3 CV |
| Gradient | BufferA: Water | 4 to 5 | 19 CV |
| Elution | Buffer B: 400 mM | | |
| | Phosphate, pH 6.8 from | | |
| | 10% to 24% B | | |
| Strip | 400 mM Phosphate, pH 6.8 | 4 to 5 | 3 CV |
| Sanitization | 0.5N Sodium Hydroxide | 4 to 5 | 3 CV and |
| | | | 0.5 hr hold |
| Storage | 0.1N Sodium Hydroxide | 4 to 5 | 3 CV |

TABLE 15
SE HPLC analysis of LMW
SE-HPLC Analysis

| Sample | Main Peak Purity (%) | (%) LMW Variants |
|---|---|---|
| CHT IP | 99.45 | 0.40 |
| CHT OP | 99.94 | 0.02 |

TABLE 16
CEX HPLC-BV analysis
CEX-HPLC Analysis

| Sample | Main Peak Purity (%) | (%) Basic Variants |
|---|---|---|
| CHT IP | 67.47 | 11.40 |
| CHT OP | 74.74 | 3.96 |

TABLE 17
Analysis of LMWs by CE-SDS

| | DSP Step | | | | | | | |
|---|---|---|---|---|---|---|---|---|
| | % LMW | | | | | | | |
| | 8 | 7 | 6 | 5 | 4 | 3 | Total of LMW | Main Peak |
| | LC | NA | HC | HH | HH | 2H1L | NA | Main peak |
| | ~23.9 kDa | NA | ~50.7 kDa | ~101.4 kDa | ~101.4 kDa | ~125.3 kDa | NA | ~149.2 kDa |
| AEX | 0.46 | 0 | 0.3 | 0.05 | 0.16 | 2.47 | 3.82 | 96.20 |
| Pool | | | | | | | | |
| CHT | 0.32 | 0 | 0.16 | 0.04 | 0.08 | 1.32 | 2.26 | 97.74 |
| Eluate | | | | | | | | |
| (Pool) | | | | | | | | |

Result: Table shows total reduction of LMWs by 40%.

### Example 6—Purification of Monoclonal Antibody by Performing Affinity Column Followed by Anion Exchange Column (200 L Scale)

All chromatographic processes were carried out using an AKTA Pilot or AKTA Process from GE Healthcare. Concentration of protein samples were determined by measuring absorbance at 280 nm using Spectrophotometer. Mabselect Sure LX resin media obtained from GE Healthcare. Chromatographic 250/250 columns were obtained from Millipore Corporation or GE Healthcare or Repligen. All Chemicals used during manufacturing process were of GMP grade.

A monoclonal antibody molecule capable to bind to IgE molecule expressed in Chinese Hamster Ovary (CHO) cell line is captured using Protein A (Mab Select Sure LX, GE Healthcare) packed in Chromatographic 250/250 column. The residence time is 4 min for all the phases. After equilibration with Tris HCl+150 mM NaCl, pH 6.8-7.2 clarified harvest is loaded at ≤45 mg/mL of the resin. After loading column is washes with equilibration buffer (wash 1) followed by wash 2 and wash 3 buffer as mentioned in Table 18. Affinity chromatography step is operated in bind and eluate mode and collection is done from 0.25 AU/cm ascending to 0.25 AU/cm descending of the peak. Protein A output is analysed with SE-HPLC, CEX-HPLC for size and charge variants. The experimental design for Protein A step is summarized in Table 18.

TABLE 18
Experimental design for Protein A

| | | Residence | Column |
|---|---|---|---|
| | | Time | Volume |
| Step | Buffer | (min) | (CV) |
| Sanitization | 0.1M NaOH | 4 | 2-3 |
| Equilibration | 20 mM Tris-HCl + 150 mM | 4 | 5 or to pH |
| | NaCl, pH 7.0 ± 0.2 | | end point |
| Load | Clarified Harvest | 4 | Till loading |
| | | | volume |
| Wash 1 | 20 mM Tris-HCl + 150 mM | 4 | 3 |
| | NaCl, pH 7.0 ± 0.2 | | |
| Wash 2 | 20 mM Tris-HCl + 1M NaCl, | 4 | 5 |
| | pH 7.0 ± 0.2 | | |
| Wash 3 | 30 mM Sodium acetate, pH | 4 | 7 |
| | 5.5 ± 0.2 | | |
| Elution | 200 mM Sodium acetate, pH | 4 | 5 |
| | 3.5 ± 0.1 | | |
| Strip | 100 mM Acetic acid | 4 | 3 |
| Equilibration | 20 mM Tris-HCl + 150 mM | 4 | 3 |
| | NaCl, pH 7.0 ± 0.2 | | |
| Sanitization | 0.1M NaOH | 4 | 3 |
| Equilibration | 20 mM Tris-HCl + 150 mM | 4 | 5 or to pH |
| | NaCl, pH 7.0 ± 0.2 | | end point |
| Storage | 20% Ethanol | 4 | 3 |

Eluted protein is further purified using Anion Exchange Chromatography resin (POROS 50 HQ, Thermofisher) packed in Chromatographic 350/250 or 200/350 column. The residence time is 4 min for all the phases. After equilibration with Tris HCl, pH 7.1-7.3 Neutralized 5 Protein A output is loaded at 70 to 145 mg/mL of the resin. The Load is diluted with water to meet the (mS/cm) conductivity specification i. e. ≤2.0 mS/cm before introducing it into the AEX column.

AEX step is operated in Flow-through (negative) mode and collection is done from 2.0 AU/cm ascending to 2.5 AU/cm descending of the peak. The column is washed using Tris HCl, pH 7.1 10-7.3. AEX output is analysed with SE-HPLC, CEX-HPLC for size and charge variants. The experimental design for POROS 50 HQ step is summarized in Table 19.

TABLE 19
Experimental design for AEX:

| | | Residence | |
|---|---|---|---|
| | | Time | Column Volume |
| Step | Buffer | (min) | (CV) |
| Sanitization | 0.5N Sodium Hydroxide | 4 | 3 CV |
| Charge | 20 mM Tris HCl, 1M | 4 | 5 CV or pH end |
| | NaCl pH 7.2 | | point |
| Equilibration | 20 mM Tris HCl, pH 7.2 | 4 | 5 CV or pH end |
| | | | point |
| Load | AEX Load pH 7.2 | 4 | Till loading volume |
| | conductivity ≤ 2.0 | | |
| | mS/cm | | |
| Chase | 20 mM Tris HCl, pH 7.2 | 4 | 3 CV |
| Strip | 20 mM Tris HCl, 1M | 4 | 3 CV |
| | NaCl pH 7.2 | | |
| Sanitization | 0.5N Sodium Hydroxide | 4 | 3 CV |
| Storage | 0.1N Sodium Hydroxide | 4 | 3 CV |

TABLE 20
SE (Size Exclusion)-HPLC (High performance Liquid
Chromatography Analysis)
SE-HPLC Analysis

| Sample | Main Peak Purity (%) | HMW (%) |
|---|---|---|
| AEX IP | 95.80 | 3.71 |
| AEX OP | 99.30 | 0.32 |

Table 20 shows 99% purity of main peak and 91% reduction of HMW which is determined by SE-HPLC.

CEX-HPLC Analysis

| Sample | Main Peak (%) | Acidic Variants (%) |
|---|---|---|
| NPEL/AEX IP | 69.34 | 15.90 |
| AEX OP | 74.19 | 8.50 |

Table 21 shows 74% purity of main peak and approximately 46% reduction of acidic variants which is determined by CEX-HPLC.

### Example 7—Purification of Monoclonal Antibody by Performing Affinity Chromatography Followed by AEX Chromatography Followed by CHT XT Resin (for 200 L Scale)

All chromatographic processes were carried out using an AKTA Pilot or AKTA Process system from GE Healthcare. Concentration of protein samples were determined by measuring absorbance at 280 nm using Spectrophotometer. CHT XT resin media obtained from Bio-rad. Chromatographic 300/250 columns were obtained from Millipore Corporation or GE Healthcare or Repligen. All Chemicals used during manufacturing process were of GMP grade. A monoclonal antibody capable to bind to IgE expressed in Chinese Hamster Ovary (CHO) cell line is captured using Protein A (Mab Select Sure LX, GE Healthcare) packed in Chromatographic 250/250 column. Eluted protein is further subjected to viral inactivation and neutralization followed by depth filtration. After depth filtration, protein has been filtered by 0.2 μm filter.

Post depth filtration step, the protein mixture is further purified using Anion Exchange Chromatography resin (Poros HQ, Thermofisher) packed in Chromatographic 350/250 or 200/250 column.

Eluted protein from Anion Exchange Chromatography (AEX) is further polished using mixed-mode resin (CHT XT, Bio-rad) packed in Chromatographic 300/250 column. The residence time is 4 min/5 min for all the phases. After equilibration with Na Phosphate, pH 6.8 Anion exchange (AEX) output is loaded at maximum of 12.5 mg/mL of the resin. The column is washed using 3 CV, Na Phosphate, pH 6.8. Bound protein of interest is eluted using linear gradient between 40 mM Na Phosphate and 400 mM Na Phosphate, pH 6.8. Eluted peak of interest is analysed with SE-HPLC, CEX-HPLC for size and charge variants. The experimental design and process for CHT XT step is summarized in Table 22-24.

TABLE 22
Experimental design for CHT XT

| | | Residence | Column |
|---|---|---|---|
| | | Time | Volume |
| Step | Buffer | (min) | (CV) |
| Sanitization | 0.5N Sodium Hydroxide | 4 to 5 | 3 CV and |
| | | | 0.5 hr hold |
| Charge | 400 mM Na Phosphate, | 4 to 5 | 3 CV |
| | pH 6.8 | | |
| Equilibration | 40 mM Na Phosphate, | 4 to 5 | 3 CV or |
| | pH 6.8 | | conductivity |
| | | | end point |
| Load | AEX Eluate + 40 mM | 4 to 5 | Till loading |
| | Na Phosphate, pH 6.8 | | volume |
| Wash | 40 mM Na Phosphate, | 4 to 5 | 3 CV |
| | pH 6.8 | | |
| Gradient | Buffer A: Water | 4 to 5 | 19 CV |
| Elution | Buffer B: 400 mM | | |
| | Phosphate, pH 6.8 from | | |
| | 10% to 24% B | | |
| Strip | 400 mM Phosphate, | 4 to 5 | 3 CV |
| | pH 6.8 | | |
| Sanitization | 0.5N Sodium Hydroxide | 4 to 5 | 3 CV and |
| | | | 0.5 hr hold |
| Storage | 0.1N Sodium Hydroxide | 4 to 5 | 3 CV |

TABLE 23
SE HPLC analysis of LMW
SE-HPLC Analysis

| Sample | Main Peak Purity (%) | (%) LMW Variants |
|---|---|---|
| CHT IP | 99.30 | 0.43 |
| CHT OP | 99.90 | 0.01 |

TABLE 24
CEX HPLC-BV analysis
CEX-HPLC Analysis

| Sample | Main Peak Purity (%) | (%) Basic Variants |
|---|---|---|
| CHT IP | 74.19 | 10.80 |
| CHT OP | 81.11 | 2.70 |

### Example 8—Purification of Monoclonal Antibody Using Strong Anion Exchange (500-Liter Batch)

5 batches were taken at 500 L scale and all the 5 batches; batch 1 to batch 5 were performed according to the process given below:

An Omalizumab monoclonal antibody molecule expressed in the Chinese Hamster Ovary (CHO) cell line is captured using Protein A (Mab Select Sure LX, GE Healthcare) packed in BPG 300/500 column.

Eluted protein is further subjected to viral inactivation and neutralization. After neutralization, protein has been filtered by 0.2 μm filter.

Post viral inactivation and neutralization step, eluted protein is further purified using Anion Exchange Chromatography resin (POROS 50 HQ, Thermofisher) packed in Chromatographic 300/500 column. The residence time is 4 min for all the phases. After equilibration with Tris HCl, pH 7.1-7.3 Neutralized Protein A output is loaded at 70 to 145 mg/mL of the resin. The Load is diluted with water to meet the (mS/cm) conductivity specification i. e. ≤2.0 mS/cm before introducing it into the AEX column.

AEX step is operated in Flow-through (negative) mode and collection is done from 2.0 AU/cm ascending to 2.5 AU/cm descending of the peak. The column is washed using Tris HCl, pH 7.1-7.3. AEX output is analyzed with SE-HPLC, CEX-HPLC for size and charge variants. The experimental design for POROS 50 HQ step is summarized in Table 25.

TABLE 25
Experimental design for AEX

| | | Residence | Column Volume |
|---|---|---|---|
| Step | Buffer | Time (min) | (CV) |
| Sanitization | 0.5N Sodium Hydroxide | 4 | 3 CV |
| Charge | 20 mM Tris HCl, 1M NaCl | 4 | 5 CV or pH end point |
| | pH 7.2 | | |
| Equilibration | 20 mM Tris HCl, pH 7.2 | 4 | 5 CV or pH end point |
| Load | AEX Load pH 7.2 | 4 | Till loading volume |
| | conductivity ≤2.0 mS/cm | | |
| Chase | 20 mM Tris HCl, pH 7.2 | 4 | 3 CV |
| Strip | 20 mM Tris HCl, 1M NaCl | 4 | 3 CV |
| | pH 7.2 | | |
| Sanitization | 0.5N Sodium Hydroxide | 4 | 3 CV |
| Storage | 0.1N Sodium Hydroxide | 4 | 3 CV |

TABLE 26
SE (Size Exclusion)-HPLC (High-performance Liquid Chromatography Analysis)
Main peak

| Stage | Batch 1 | Batch 2 | Batch 3 | Batch 4 | Batch 5 | Min | Max | Avg |
|---|---|---|---|---|---|---|---|---|
| AEX Load | 96.00 | 95.50 | 96.40 | 95.70 | 95.50 | 95.50 | 96.40 | 95.82 |
| AEX OP | 99.20 | 98.40 | 99.10 | 99.50 | 99.70 | 98.40 | 99.70 | 99.18 |

high molecular weight species (HMW)

| | Batch 1 | Batch 2 | Batch 3 | Batch 4 | Batch 5 | Min | Max | Avg |
|---|---|---|---|---|---|---|---|---|
| AEX Load | 3.60 | 3.20 | 2.80 | 3.20 | 3.20 | 2.80 | 3.60 | 3.20 |
| AEX OP | 0.40 | 0.40 | 0.30 | 0.40 | 0.20 | 0.20 | 0.40 | 0.34 |

Ratio: HMW/Main peak

| Pool | Batch 1 | Batch 2 | Batch 3 | Batch 4 | Batch 5 | Min | Max | Avg |
|---|---|---|---|---|---|---|---|---|
| AEX Load | 0.0375 | 0.0335 | 0.0290 | 0.0334 | 0.0335 | 0.0290 | 0.0375 | 0.0334 |
| AEX OP | 0.0040 | 0.0041 | 0.0030 | 0.0040 | 0.0020 | 0.0020 | 0.0041 | 0.0034 |

Table 26 shows 99% purity of the main peak, 89% reduction of HMW and 89.8% reduction of average ratio of HMW to main peak, as determined by SE-HPLC.

TABLE 27
shows the results of Cation Exchange-High-performance Liquid
Chromatography comparing the main peak with acidic variants.

| | | | | | | | | |
|---|---|---|---|---|---|---|---|---|
| Pool | Batch 1 | Batch 2 | Batch 3 | Batch 4 | Batch 5 | Min | Max | Avg |

Main peak by CEX-HPLC

| | | | | | | | | |
|---|---|---|---|---|---|---|---|---|
| AEX Load | 74.50 | 72.30 | 74.40 | 74.80 | 74.70 | 72.30 | 74.80 | 74.14 |
| AEX OP | 76.80 | 76.00 | 76.80 | 77.80 | 76.40 | 76.00 | 77.80 | 76.76 |

Acidic variants by CEX-HPLC

| | | | | | | | | |
|---|---|---|---|---|---|---|---|---|
| AEX Load | 13.80 | 15.00 | 13.60 | 13.50 | 13.80 | 13.50 | 15.00 | 13.94 |
| AEX OP | 11.10 | 11.10 | 10.80 | 10.50 | 11.00 | 10.50 | 11.10 | 10.90 |

Ratio: AV/Main peak

| | | | | | | | | |
|---|---|---|---|---|---|---|---|---|
| AEX Load | 0.19 | 0.21 | 0.18 | 0.18 | 0.18 | 0.18 | 0.21 | 0.19 |
| AEX OP | 0.14 | 0.15 | 0.14 | 0.13 | 0.14 | 0.13 | 0.15 | 0.14 |

Table 27 shows the reproducibility and consistency of five batches pf omalizumab at 500 L where main peak and acidic variants remains in desirable amount and maintain the average ratio. 76% purity of the main peak, 21% reduction of acidic variants and 26% reduction of average ratio of acidic variants to main peak, as determined by CEX-HPLC.

### Example 9—Purification of Monoclonal Antibody by Performing Affinity Chromatography Followed by AEX Chromatography Followed by CHT XT Resin (for 500 L Scale)

5 batches were taken at 500 L scale and all the 5 batches; batch 1 to batch 5 were performed according to the process given below:

An Omalizumab monoclonal antibody molecule expressed in the Chinese Hamster Ovary (CHO) cell line is captured using Protein A (Mab Select Sure LX, GE Healthcare) packed in BPG 300/500 column.

Eluted protein is further subjected to viral inactivation and neutralization. After neutralization, protein has been filtered by 0.2 μm filter.

Post viral inactivation and neutralization step, eluted protein is further purified using Anion Exchange Chromatography resin (POROS 50 HQ, Thermofisher) packed in Chromatographic 300/500 column. The residence time is 4 min for all the phases.

Eluted protein from Anion Exchange Chromatography (AEX) is further polished using mixed-mode resin (CHT XT, Bio-rad) packed in Chromatographic 300/250 column. The residence time is 4 min/5 min for all the phases. After equilibration with Na Phosphate, pH 6.8 Anion exchange (AEX) output is loaded at maximum of 12.5 mg/mL of the resin. The column is washed using 3 CV, Na Phosphate, pH 6.8. Bound protein of interest is eluted using linear gradient between 40 mM Na Phosphate and 400 mM Na Phosphate, pH 6.8. Eluted peak of interest is analysed with SE-HPLC, CEX-HPLC for size and charge variants. The experimental design and process for CHT XT step is summarized in Table 28-30.

TABLE 28
Experimental design for CHT XT

| | | Residence | Column |
|---|---|---|---|
| | | Time | Volume |
| Step | Buffer | (min) | (CV) |
| Sanitization | 0.5N Sodium Hydroxide | 4 to 5 | 3 CV and |
| | | | 0.5 hr hold |
| Charge | 400 mM Na Phosphate, | 4 to 5 | 3 CV |
| | pH 6.8 | | |
| Equilibration | 40 mM Na Phosphate, | 4 to 5 | 3 CV or |
| | pH 6.8 | | conductivity |
| | | | end point |
| Load | AEX Eluate + 40 mM | 4 to 5 | Till loading |
| | Na Phosphate, pH 6.8 | | volume |
| Wash | 40 mM Na Phosphate, | 4 to 5 | 3 CV |
| | pH 6.8 | | |
| Gradient | Buffer A: Water | 4 to 5 | 19 CV |
| Elution | Buffer B: 400 mM | | |
| | Phosphate, pH 6.8 from | | |
| | 10% to 24% B | | |
| Strip | 400 mM Phosphate, | 4 to 5 | 3 CV |
| | pH 6.8 | | |
| Sanitization | 0.5N Sodium Hydroxide | 4 to 5 | 3 CV and |
| | | | 0.5 hr hold |
| Storage | 0.1N Sodium Hydroxide | 4 to 5 | 3 CV |

TABLE 29
shows the results of SE (Size Exclusion)-HPLC (High-performance Liquid
Chromatography Analysis) comparing the main peak with LMW species
Main peak

| Stage | Batch 1 | Batch 2 | Batch 3 | Batch 4 | Batch 5 | Min | Max | Avg |
|---|---|---|---|---|---|---|---|---|
| CHT Load | 99.2 | 98.4 | 99.1 | 99.5 | 99.7 | 98.4 | 99.7 | 99.18 |
| CHT OP | 99.7 | 99.9 | 99.9 | 99.8 | 99.9 | 99.7 | 99.9 | 99.84 |

low molecular weight species (LMW)

| | Batch 1 | Batch 2 | Batch 3 | Batch 4 | Batch 5 | Min | Max | Avg |
|---|---|---|---|---|---|---|---|---|
| CHT Load | 0.4 | 1.2 | 0.6 | 0.1 | 0.1 | 0.1 | 1.2 | 0.48 |
| CHT OP | 0.2 | 0.0 | 0.0 | 0.1 | 0.1 | 0.0 | 0.2 | 0.08 |

Ratio: LMW/Main peak

| Pool | Batch 1 | Batch 2 | Batch 3 | Batch 4 | Batch 5 | Min | Max | Avg |
|---|---|---|---|---|---|---|---|---|
| CHT Load | 0.0040 | 0.012 | 0.0060 | 0.001 | 0.001 | 0.001 | 0.0060 | 0.0048 |
| CHT OP | 0.002 | 0 | 0 | 0.001 | 0.001 | 0.0 | 0.002 | 0.0008 |

Table 29 shows the reproducibility and consistency of five batches pf omalizumab at 500 L where main peak and LMWs remains in desirable amount and maintain the average ratio. 99% purity of the main peak, 50-100% reduction in LMW and 50-100% reduction of average ratio of LMW to main peak, as determined by SE-HPLC.

TABLE 30
shows the results of Cation Exchange-High-performance Liquid
Chromatography comparing the main peak with Basic variants.

| | | | | | | | | |
|---|---|---|---|---|---|---|---|---|
| Pool | Batch 1 | Batch 2 | Batch 3 | Batch 4 | Batch 5 | Min | Max | Avg |

Main peak by CEX—HPLC

| | | | | | | | | |
|---|---|---|---|---|---|---|---|---|
| CHT Load | 76.8 | 76.0 | 76.8 | 77.8 | 76.4 | 76.4 | 77.8 | 76.76 |
| CHT OP | 83.6 | 84.9 | 85.2 | 84.3 | 83.5 | 83.5 | 85.2 | 84.3 |

Basic variants by CEX—HPLC

| | | | | | | | | |
|---|---|---|---|---|---|---|---|---|
| CHT Load | 12.2 | 12.9 | 12.4 | 11.7 | 12.5 | 11.7 | 12.9 | 12.34 |
| CHT OP | 4.5 | 3.7 | 4.2 | 4.1 | 4.8 | 3.7 | 4.8 | 4.26 |

Ratio: BV/Main peak

| | | | | | | | | |
|---|---|---|---|---|---|---|---|---|
| CHT Load | 0.1588 | 0.1697 | 0.1614 | 0.1503 | 0.1636 | 0.1503 | 0.1697 | 0.1607 |
| CHT OP | 0.0538 | 0.0435 | 0.0492 | 0.0492 | 0.0575 | 0.0435 | 0.575 | 0.0506 |

Table 30 shows the reproducibility and consistency of five batches pf omalizumab at 500 L where main peak and basic variants remains in desirable amount and maintain the average ratio. 84% purity of the main peak, 65% reduction of basic variants and 68% reduction of average ratio of basic variants to main peak, as determined by CEX-HPLC.

### Example 10: Process of Preparing Drug Substance

The drug substance was produced by a downstream purification process comprising Protein A capture chromatography, followed by low pH virus inactivation and subsequent neutralization, depth filtration, anion exchange chromatography (AEX), ceramic hydroxyapatite chromatography (CHT), and viral filtration (VF) preferably using a 0.2μ filter thereby generating a viral filtrate.

The viral filtrate was concentrated subjecting to a tangential flow filtration using a suitable semipermeable membrane, Millipore Pellicon® 3, Biomax® A screen, 30 kDa NMWCO membrane, which was further subjected to concentration through ultrafiltration/diafiltration (UFDF) and buffer exchange with the exchange buffer having composition of 200 mM Arginine and 20 mM phosphate at pH 6, followed by final concentration.

### Example 11

The method of evaluation and results of percentage of high molecular weight variants (HMW), low molecular weight variants (LMW) variants, LC (light chain) and HC (heavy chain) of biosimilar of omalizumab for 30 days stability at 5 °C±3 °C (mentioned in table 31)

TABLE 31
Percentage of High Molecular Weight Variants (HMW), Low Molecular
Weight Variants (LMW) variants, LC (light chain) and HC (heavy chain) of biosimilar
of omalizumab for 30 days stability at 5 °C ± 3 °C

| | | Time Interval (Days) | | | | | |
|---|---|---|---|---|---|---|---|
| | | | Initial | | | | |
| | Acceptance | | (0 | | | 15 | |
| Test/Method | Criteria | Batches | days) | 3 days | 7 days | days | 30 days |
| Purity By Size | | B1 | 0.18 | 0.16 | 0.21 | 0.30 | 0.35 |
| Exclusion | | B2 | 0.09 | 0.09 | 0.12 | 0.12 | 0.21 |
| | | B3 | 0.11 | 0.07 | 0.07 | 0.09 | 0.12 |
| Chromatography | High Molecular | B4 | 0.06 | 0.13 | 0.12 | 0.18 | 0.20 |
| (SEC) | Weight | | | | | | |
| | Variants: | B5 | 0.07 | 0.07 | 0.06 | 0.09 | 0.09 |
| | NMT 0.50% | | | | | | |
| CE-SDS | Low Molecular | B1 | 2.2 | 2.4 | 1.3 | 1.5 | 1.5 |
| Non-Reduced | Weight | B2 | 1.8 | 2.2 | 2.7 | 1.7 | 2.1 |
| | Variants: NMT | B3 | 3.43 | 2.8 | 3.0 | 2.9 | 2.7 |
| | 5.0% | B4 | 2.7 | 2.2 | 2.5 | 2.4 | 2.2 |
| | | B5 | 1.8 | 1.4 | 1.3 | 2.2 | 1.5 |
| CE-SDS Reduced | % LC: NLT | B1 | 31 | 32 | 32 | 32 | 31 |
| | 26% | B2 | 31 | 31 | 31 | 31 | 31 |
| | | B3 | 31 | 31 | 31 | 31 | 31 |
| | | B4 | 32 | 31 | 31 | 31 | 31 |
| | | B5 | 31 | 31 | 31 | 31 | 32 |
| | %HC: NLT | B1 | 67 | 67 | 67 | 67 | 67 |
| | 63% | B2 | 66 | 67 | 67 | 67 | 67 |
| | | B3 | 66 | 66 | 66 | 66 | 66 |
| | | B4 | 67 | 67 | 68 | 68 | 67 |
| | | B5 | 67 | 67 | 67 | 67 | 67 |

Based on the above result mentioned in table 31, it can be concluded that the biosimilar of omalizumab is stable at 5 °C±3 °C for 30 days in all 5 batches. Hence there is stability and consistency across all five batches.

### Example 12: Details of Column Parameters, Sample Solution Preparation and Gradients Used in High Performance-Cation Exchange Chromatography (HP-CEX)

Sample of biosimilar of omalizumab was run through HP-CEX (High performance-Cation exchange chromatography) for analysis or characterization. The injection volume was taken 25.00 μl and run time was 75.0 Minutes.

All the experiments were performed on liquid chromatography system using following parameters and gradients:

TABLE 32
Parameters for High performance - Cation exchange chromatography
(HP-CEX)

| | |
|---|---|
| HPLC system | HPLC system equipped with the pump, an autosampler, |
| | a UV detector and a suitable data acquisition system |
| Column | Thermo Scientific ™, cation exchange (CEX) |
| | ProPac ™ Elite WCX, |
| | 4 mm × 250 mm, 5 μm, part number 303025 or |
| | equivalent |
| Mobile phase | Mobile phase A: 20 mM MES, pH 6.5 |
| | Mobile phase B: 20 mM MES, 200 mM NaCl, pH 6.5 |
| Detection | UV at 280 nm |
| Flow rate | 0.6 mL/min |
| Injection volume | 25 μL |
| Injection Amount | 50 μg¹ |
| Column | 40 °C |
| Temperature | |
| Sample | 6 ºC ± 1 °C |
| temperature | |
| Quantitation | Peak area response |
| Run Time | 75 minutes (Main peak observed between 24 to |
| | 40 minutes) |
| Needle wash | 5% (v/v) methanol in water or diluent |

TABLE 33
Gradient details for High performance - Cation exchange
chromatography (HP-CEX)

| Time (minutes) | Mobile phase A (%) | Mobile phase B (%) |
|---|---|---|
| 0 | 90 | 10 |
| 2 | 90 | 10 |
| 47 | 72 | 28 |
| 48 | 0 | 100 |
| 53 | 0 | 100 |
| 54 | 90 | 10 |
| 75 | 90 | 10 |

### Example 13: Types of Acidic and Basic Variants that are Present in Biosimilar of Omalizumab

Acidic charge variant such as deamidated, sialylated glycans and basic charge variant such as Succinimide variant and 1× lysine variant & 2× lysine variant are well detected and details are as follows:

TABLE 34
Types of acidic and basic variants

| | |
|---|---|
| Main peak | Biosimilar of omalizumab intact without any modification |
| Acidic variants | Presence of multiple deamidated variant, glycated variant, |
| | sialylated glycans |
| Basic variants | Succinimide variant, 1x lysine variant & 2x lysine variant |

### Example 14: Analysis of Charged Variants by Cation Exchange Chromatography (CEX) and Stability Report of Biosimilar of Omalizumab at 150 mg/ml Drug Substance (PFS)

The stability analysis was conducted for different time periods at different temperature conditions. The stability study was performed under stress conditions at 5 °C±3 °C for 30 days and samples were analysed on day 0, 3, 7 and 15.

The purpose of the study was the establishment of the shelf life of the drug substance. The results are mentioned in below tables 32 where percent of main peak including K1/K2 peaks, acidic charge variants and basic charge variants excluding K1/K2 peaks present in biosimilar of omalizumab are mentioned at respective time periods which is maintained in five batches.

TABLE 35
Results of percent of main peak including K1/K2 peaks, acidic charge variants
and basic charge variants excluding K1/K2 peaks of biosimilar of omalizumab for 30
days.

| | | Time Interval (Days) | | | | | |
|---|---|---|---|---|---|---|---|
| | Acceptance | | Initial (0 | | | 15 | |
| Test/Method | Criteria | Batches | days) | 3 days | 7 days | days | 30 days |
| Charged Variants | Main Peak | B1 | 83.6 | 84.0 | 84.0 | 83.9 | 83.6 |
| by Cation | including K1/ | B2 | 83.9 | 83.9 | 84 | 83.2 | 82.6 |
| Exchange | K2 peaks: NLT | B3 | 83.3 | 83 | 83 | 83.3 | 82 |
| Chromatography | 75.0% | B4 | 84.1 | 83.5 | 83 | 82.8 | 82.3 |
| (CEX) | | B5 | 82.8 | 82.4 | 80.9 | 82.5 | 81 |
| | Acidic Charge | B1 | 10.2 | 9.9 | 9.8 | 9.9 | 9.8 |
| | Variants: | B2 | 10.8 | 10.8 | 10.7 | 10.6 | 10.6 |
| | NMT 13.0% | B3 | 9.4 | 9.4 | 9.3 | 9.2 | 9.5 |
| | | B4 | 9.5 | 9.8 | 9.4 | 9.4 | 9.3 |
| | | B5 | 11.6 | 11.7 | 11.8 | 11.7 | 11.4 |
| | Basic Charge | B1 | 6.2 | 6.2 | 6.1 | 6.2 | 6.6 |
| | Variants | B2 | 5.3 | 5.3 | 5.3 | 6.2 | 6.8 |
| | excluding K1/ | B3 | 7.1 | 7 | 7.3 | 7.2 | 7.9 |
| | K2 peaks: | B4 | 6.1 | 6.4 | 7.2 | 7.4 | 7.9 |
| | NMT 12.0% | B5 | 5.5 | 6.0 | 7.3 | 5.9 | 7.6 |

Based on the above result mentioned in table 32, it can be concluded that the biosimilar of omalizumab is stable at 5 °C±3 °C for 30 days and −70 °C±10 °C for up to 24 months in all 5 batches. Hence there is stability and consistency across all five batches.

That which is claimed is:

1. A drug substance composition of omalizumab or a biosimilar of omalizumab comprising:
a monomer and one or more impurities selected from high molecular weight impurities (HMW) and low molecular weight impurities (LMW) wherein;
a) the high molecular weight impurities are present in an amount less than about 1% as analyzed by size exclusion chromatography (SEC),
b) the low molecular weight impurities are present in an amount less than about 5% as analyzed by non-reduced capillary electrophoresis sodium dodecyl sulfate (CE-SDS);
wherein, the monomer of omalizumab or a biosimilar of omalizumab has a purity at least more than 90% as analyzed by reduced size exclusion chromatography (rSEC);
wherein, the drug substance is stable when stored at about 2 °C to about 8 °C for a suitable period;
wherein, the suitable period is selected from three days, seven days, fifteen days and thirty days.

2. The drug substance composition according to claim 1, wherein the monomer of omalizumab or a biosimilar of omalizumab comprising a light chain and a heavy chain in a suitable amount comprising;
a) the light chain is present in an amount of not less than 26% as determined by reduced capillary electrophoresis sodium dodecyl sulfate (CE-SDS); and
b) the heavy chain is present in an amount of not less than 63% as determined by reduced capillary electrophoresis sodium dodecyl sulfate (CE-SDS).

3. The drug substance composition according to claim 1, wherein the high molecular weight impurities (HMW) are present in an amount of about 0.95%, about 0.9%, about 0.85%, about 0.80%, about 0.75%, about 0.70%, about 0.65%, about 0.60%, about 0.55%, about 0.50%, about 0.45%, about 0.40%, about 0.35%, about 0.30%, about 0.25%, about 0.21%, about 0.20%, about 0.18%, about 0.16%, about 0.15%, or about 0.1%.

4. The drug substance composition according to claim 1, wherein the low molecular weight impurities (LMW) are present in an amount of about 5%, about 4%, about 3%, about 2.9%, about 2.8%, about 2.7%, about 2.6%, about 2.5%, about 2.4%, about 2.3%, about 2.2%, about 2.1%, about 2.0%, about 1.9%, about 1.8%, about 1.7%, about 1.6%, about 1.5%, about 1.4%, about 1.3%, about 1.2%, about 1.1%, about 1.0%, about 0.9%, about 0.8%, about 0.7%, about 0.6%, or about 0.5%.

5. The drug substance composition according to claim 1, wherein the composition maintains the stability in at least four batches.

6. The drug substance composition according to claim 5, wherein the batches are performed at scale of 50 L, 200 L 500 L, 1000 L, 2000 L, or 5000 L.

7. A drug substance composition of omalizumab or a biosimilar of omalizumab comprising:
a monomer omalizumab or a biosimilar of omalizumab and one or more impurities selected from acidic variants and basic variants wherein;
a) the acidic variants are present in an amount of below 13%; and
b) the basic variants are present in an amount of below 10%;
wherein, the acidic variants and the basic variants are measured by high performance-cation exchange chromatography (HP-CEX) analysis by UV detector;
wherein, the drug substance is stable at about 2 °C to about 8 °C for a suitable period;
wherein, the suitable period is selected from three days, seven days, fifteen days and thirty days.

8. The drug substance composition according to claim 7, wherein the monomer of omalizumab or a biosimilar of omalizumab comprising a light chain and a heavy chain in a suitable amount comprising;
a) the light chain present in an amount of not less than 26% as determined by reduced capillary electrophoresis sodium dodecyl sulfate (CE-SDS); and
b) the heavy chain present in an amount of not less than 63% as determined by reduced capillary electrophoresis sodium dodecyl sulfate (CE-SDS).

9. The drug substance composition according to claim 7 wherein,
the acidic variants are present in an amount of below 11%;
the acidic variants are present in an amount of selected from 10.2%, 10%, 9.9%, 9.8%, 9.5%, 9.2%;
the acidic variants are present in an amount of below 9%;
the acidic variants are present in an amount of below about 8%;
the acidic variants are present in an amount of below about 7%; and
the acidic variants are present in an amount of below about 6%.

10. The drug substance composition according to claim 7 wherein,
the basic variants are present in an amount of below 9%;
the basic variants are present in an amount of below about 8%;
the basic variants are present in an amount of selected from 7%, 6.6%, 6.2%, 6.1%;
the basic variants are present in an amount of below about 6%;
the basic variants are present in an amount of below about 5%; and
the basic variants are present in an amount of below about 4%.

11. The drug substance composition according to claim 9 and claim 10 wherein,
a) the acidic variants are selected from deamidated variants, glycated variants and sialylated variants; and
b) the basic variants are selected from a succinimide variants, 1× lysine variants (K1), 2× lysine variants (K2), isoaspartate variants, isoimide variants, oxidated variant and amidated variants.

12. The drug substance composition according to claim 2 and claim 8, wherein the light chain is present in an amount of about 26%, about 27%, about 28%, about 29%, about 30%, about 31%, about 32%, about 33%, about 34% or about 35%.

13. The drug substance composition according to claim 2 and claim 8, wherein the heavy chain is present in an amount of about 63%, about 64%, about 65%, about 66%, about 67%, about 68%, about 69%, about 70%, about 71%, about 72%, about 73%, about 74% or about 75%.

14. The drug substance composition according to claim 1 and claim 7, wherein the monomer of omalizumab or biosimilar of omalizumab is about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8% or about 99.9%, measured by reduced size exclusion chromatography (SEC).
